SEQUENCE LISTING (49 sequences)

SEQ ID NO: 1 - DNA, 74 bp, Artificial Sequence
  Synthetic Construct
cgtccgtatgacgagagaagctgaccagagaaacacacgacgtaagtcgtggtacattacctggtaacagaggc

SEQ ID NO: 2 - DNA, 69 bp, Artificial Sequence
  Synthetic Construct
tgttgttgttgttgttgttgtgcctatgtctcagcctctgttaccaggtaatgtaccacgacttacgtc

SEQ ID NO: 3 - DNA, 51 bp, Artificial Sequence
  Synthetic Construct
taatacgactcactatagggcagccgtcctcgtccgtatgacgagagaagc

SEQ ID NO: 4 - DNA, 45 bp, Artificial Sequence
  Synthetic Construct
tatgacgaggacggctggttgttgttgttgttgttgttgttgtgc

SEQ ID NO: 5 - DNA, 45 bp, Artificial Sequence
  Synthetic Construct
tatgacgaggacggctgattgttgttgttgttgttgttgttgtgc

SEQ ID NO: 6 - DNA, 45 bp, Artificial Sequence
  Synthetic Construct
tatgacgaggacggctgagtgttgttgttgttgttgttgttgtgc

SEQ ID NO: 7 - DNA, 45 bp, Artificial Sequence
  Synthetic Construct
tatgacgaggacggctgagagttgttgttgttgttgttgttgtgc

SEQ ID NO: 8 - DNA, 45 bp, Artificial Sequence
  Synthetic Construct
tatgacgaggacggctgagacttgttgttgttgttgttgttgtgc

SEQ ID NO: 9 - DNA, 45 bp, Artificial Sequence
  Synthetic Construct
tatgacgaggacggctgagacatgttgttgttgttgttgttgtgc

SEQ ID NO: 10 - DNA, 17 bp, Artificial Sequence
  Synthetic Construct
gcttctctcgtcatacg

SEQ ID NO: 11 - DNA, 28 bp, Artificial Sequence
  Synthetic Construct
taatacgactcactatagggcagccgtc

SEQ ID NO: 12 - DNA, 29 bp, Artificial Sequence
  Synthetic Construct
ggtgacactatgatgcatatgacgaggac

SEQ ID NO: 13 - RNA, 60 nt, Artificial Sequence
  Synthetic Construct
  misc_feature 1-5, 10, 13-19, 27-35, 41-48, 54, 56-60: n = A, U, G or C
  misc_feature 6: n = U, C or G
  misc_feature 7, 11, 49: n = U or C
  misc_feature 8, 25: n = A, C or U
  misc_feature 12: n = A or G
  misc_feature (55)...(55): n = A, C or G
nnnnnnnngnnnnnnnnnncauuanannnnnnnnncaaagnnnnnnnnngaagnnnnnnn

SEQ ID NO: 14 - RNA, 22 nt, Artificial Sequence
  Synthetic Construct
guucucuucaagggacaaggcu

SEQ ID NO: 15 - RNA, 119 nt, Artificial Sequence
  Synthetic Construct
guccuguccguaugacagagaagucaaccagagaaacacacguugugguauauuaccuggucaagagaaguucccuguuccuuuuuuucccgagccuugucccuugaagagaacugacc

SEQ ID NO: 16 - RNA, 139 nt, Artificial Sequence
  Synthetic Construct
  misc_feature (82)...(101): n = A, G, C or U
gggcagccguccucguccguaugacgagagaagcugaccagagaaacacacgacguaagucgugguacauuaccugguaacnnnnnnnnnnnnnnnnnnnngagaauaacaacaacaacaacaaccagccguccucguc

SEQ ID NO: 17 - RNA, 122 nt, Artificial Sequence
  Synthetic Construct
  misc_feature (74)...(93): n = A, G, C or U
guccucguccguaugacgagagaagcugaccagagaaacacacgacguaagucgugguacauuaccugguaacnnnnnnnnnnnnnnnnnnnngagaauaacaacaacaacaacaaccagcc

SEQ ID NO: 18 - RNA, 142 nt, Artificial Sequence
  Synthetic Construct
  misc_feature (121)...(125): n = A, C, G or U
gggcagccguccuggaccguauguccagagaagcugaccagagaaacacacgacguaagucgugguacauuaccugguaacagaggcugagacauaggcacaacaacaacaacaacaacannnnncagccguccucgucaua

SEQ ID NO: 19 - RNA, 10 nt, Artificial Sequence
  Synthetic Construct
ugucucagcc

SEQ ID NO: 20 - RNA, 10 nt, Artificial Sequence
  Synthetic Construct
agucucagcc

SEQ ID NO: 21 - RNA, 10 nt, Artificial Sequence
  Synthetic Construct
acucucagcc

SEQ ID NO: 22 - RNA, 10 nt, Artificial Sequence
  Synthetic Construct
acacucagcc

SEQ ID NO: 23 - RNA, 10 nt, Artificial Sequence
  Synthetic Construct
acaaucagcc

SEQ ID NO: 24 - RNA, 10 nt, Artificial Sequence
  Synthetic Construct
acaaccagcc

SEQ ID NO: 25 - RNA, 143 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacaacucucaagccguccucgucaua

SEQ ID NO: 26 - RNA, 134 nt, Artificial Sequence
  Synthetic Construct
guccucguccguaugacgagagaagcugaccagagaaacacacgacguaagucgugguacauuaccugguaacagaggcugagacauaggcacaacaacaacaacaacaacaacucucagccguccucgucaua

SEQ ID NO: 27 - RNA, 20 nt, Artificial Sequence
  Synthetic Construct
cugccuaugucucagccucu

SEQ ID NO: 28 - RNA, 122 nt, Artificial Sequence
  Synthetic Construct
guccucguccguaugacgagagaagcugaccagagaaacacacgacguaagucgugguacauuaccugguaacagaggcugagacauaggcacaacaacaacaacaacaacaacucucagcc

SEQ ID NO: 29 - RNA, 12 nt, Artificial Sequence
  Synthetic Construct
guccucgucaua

SEQ ID NO: 30 - RNA, 139 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacacucagccguccucgucaua

SEQ ID NO: 31 - RNA, 141 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacacucagccguccucgucauacg

SEQ ID NO: 32 - RNA, 140 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacacucagccguccucgucauac

SEQ ID NO: 33 - RNA, 138 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacacucagccguccucgucau

SEQ ID NO: 34 - RNA, 137 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacacucagccguccucguca

SEQ ID NO: 35 - RNA, 136 nt, Artificial Sequence
  Synthetic Construct
gggcagccguccucguccguaugacgagagaagcugaccagagaaacacacgacguaagucgugguacauuaccugguaacagaggcugagacauaggcacaacaacaacaacaacaacacucagccguccucguc

SEQ ID NO: 36 - RNA, 143 nt, Artificial Sequence
  Synthetic Construct
gggaggcuguccucguccguaugacgagagaagccuaccagagaaacacacgacguaagucgugguacauuaccugguaacagccuugucccuugaagagaacaacaacaacaacaacaacaacaaccaaggcuguccucguc

SEQ ID NO: 37 - RNA, 143 nt, Artificial Sequence
  Synthetic Construct
gggaggcuguccucguccguaugacgagagaagccuaccagagaaacacacgacguaagucgugguacauuaccugguacaagccuugucccuugaagagaacaacaacaacaacaacaacaacaaccaaggcuguccucguc

SEQ ID NO: 38 - RNA, 144 nt, Artificial Sequence
  Synthetic Construct
gggaggcuguccucguccguaugacgagagaagccuaccagagaaacacacgacguaagucgugguacauuaccugguacaagccuugucccuugaagagaacaacaacaacaacaacaacaacagaccaaggcuguccucguc

SEQ ID NO: 39 - RNA, 144 nt, Artificial Sequence
  Synthetic Construct
gggaggcuguccucguccguaugacgagagaagccuaccagagaaacacacgacguaagucgugguacauuaccugguaacagccuugucccuugaagagaacaacaacaacaacaacaacaacagaccaaggcuguccucguc

SEQ ID NO: 40 - RNA, 20 nt, Artificial Sequence
  Synthetic Construct
gugccuaugucucagccucu

SEQ ID NO: 41 - RNA, 122 nt, Artificial Sequence
  Synthetic Construct
guccucguccguauguccagagaagcugaccagagaaacacacgacguaagucgugguacauuaccugguaacagaggcugagacauaggcacaacaacaacaacaacaacaacacucagcc

SEQ ID NO: 42 - DNA, 20 bp, Artificial Sequence
  Synthetic Construct
gtgcctatgtctcatcctct

SEQ ID NO: 43 - DNA, 20 bp, Artificial Sequence
  Synthetic Construct
gtgcctatgtcgcatcctct

SEQ ID NO: 44 - DNA, 20 bp, Artificial Sequence
  Synthetic Construct
gtgccgatgtctcatcctct

SEQ ID NO: 45 - DNA, 20 bp, Artificial Sequence
  Synthetic Construct
gtgcctatggctcagcctct

SEQ ID NO: 46 - DNA, 20 bp, Artificial Sequence
  Synthetic Construct
gtgcctatttctcagcctct

SEQ ID NO: 47 - DNA, 20 bp, Artificial Sequence
  Synthetic Construct
gtgcctattgctcagcctct

SEQ ID NO: 48 - DNA, 20 bp, Artificial Sequence
  Synthetic Construct
gtgcttgttcctcagcctct

SEQ ID NO: 49 - RNA, 122 nt, Artificial Sequence
  Synthetic Construct
guccucguccguaugacgagagaagcugaccagagaaacacacgacguaagucgugguacauuaccugguaacagaggcugagacauaggcacaacaacaacaacaacaacaacaaccagcc

(19) United States
(12) Patent Application Publication
(10) Pub. No.: US 2007/0105108 A1
(43) Pub. Date: May 10, 2007

(54) Polynucleotides capable of target-depedent circularization and topological linkage

(75) Inventors: Sergei A. Kazakov, Los Gatos, CA (US); Anne Dallas, Santa Cruz, CA (US); Tai-Chih Kuo, Taipei (TW); Brian H. Johnston, Scotts Valley, CA (US)

Correspondence Address: MORRISON & FOERSTER LLP, 755 PAGE MILL RD, PALO ALTO, CA, 94304-1018, US

(73) Assignee: Somagenics, Inc., Santa Cruz, CA (US)

(21) Appl. No.: 10/561,691

(22) PCT Filed: Jun. 25, 2004
(86) PCT No.: PCT/US04/20589

§ 371 Date: May 30, 2006

Related U.S. Application Data
(60) Provisional application No. 60/482,653, filed on Jun. 25, 2003.

(51) Int. Cl. C40B 40/08; C40B 30/06; C07H 21/02; C07H 21/04

(52) U.S. Cl. 435/6; 536/23.1

(57) ABSTRACT

The invention provides allosterically regulatable polynucleotides capable of target-dependent circularization and topological linkage to a target nucleic acid molecule. Polynucleotides of the invention include a target binding sequence and a regulatory element which prevents circularization in the absence of the target binding. Polynucleotides may include a catalytic domain, allowing circularization to proceed via catalysis when the target binding sequence of the polynucleotide is bound to the target. Topologically linked polynucleotides may be used for detection of target molecules or to inhibit transcription or translation of the target.

### CROSS-REFERENCE TO RELATED APPLICATIONS

This application claims the benefit under 35 U.S.C. §119 of U.S. Provisional Application No. 60/482,653, filed Jun. 25, 2003, which is hereby incorporated by reference in its entirety.

### STATEMENT REGARDING FEDERALLY SPONSORED RESEARCH OR DEVELOPMENT

This invention was made in part during work supported by grant no. R43GM068225 from the National Institutes of Health.

### BACKGROUND

RNA-based technologies have become increasingly prominent in research and biotechnology since the discovery of naturally existing antisense RNAs, catalytic RNA (ribozymes), techniques for selection of aptamers from random libraries of RNA (SELEX), and RNA interference (RNAi) (Sullenger & Gilboa, 2002). A major problem for any RNA agent that relies upon efficient hybridization to complementary sequences is to identify which target sites are accessible in vivo. Consequently, the rational design of effective RNA agents can be slow and inefficient.

Over the past 25 years, modified antisense oligonucleotides (Lichtenstein & Nellen, 1998; Stein & Krieg, 1998; Crooke, 2001) and artificial ribonucleases, including ribozymes and deoxyribozymes (Rossi, 1999; Opalinska & Gewirtz, 2002; Scherer & Rossi, 2003) have been used with variable success for antisense-mediated gene silencing (ASGS) through the targeting of messenger or viral RNAs. Recently, a practical approach to the exploitation of RNAi, the use of small interfering RNAs (siRNA), has emerged (Tuschl, 2002; Zamore, 2002; Paddison & Hannon, 2003; Shi, 2003). Optimized antisense compounds and cleaving ribozymes (or DNAzymes) can work as effectively as siRNA although higher concentrations of the ASGS agents are required (Al-Anouti & Ananvoranich, 2002; Braasch & Corey, 2002; Brantl, 2002a; Opalinska & Gewirtz, 2002; Grunweller et al., 2003; Miyagishi et al., 2003; Vickers et al., 2003). A number of naturally occurring antisense RNAs that have been identified in both prokaryotes (Brantl, 2002b, Brantl & Wagner, 2002; Wagner et al., 2002) and eukaryotes (Vanhée-Brossollet & Vaquero, 1998; Kumar & Carmichael, 1998; Yelin et al., 2003), and have been shown to be highly specific and efficient in ASGS. Antisense modulation of gene expression in human cells has been suggested to be a common regulatory mechanism (Carmichael, 2003; Yelin et al., 2003). Moreover, recent results concerning the complexes formed between antisense RNA and target RNA provide direct evidence for mechanistic links between ASGS and RNAi, both of which involve double-stranded RNA (Di Serio et al., 2001; Martinez et al., 2002; Tijsterman et al., 2002; Holen et al., 2003). Artificially-designed antisense RNAs have also been proven to be powerful tools to downregulate the expression of targeted genes (including genes that are poor targets for small interference RNAs) in both prokaryotes and eukaryotes (Lafarge-Frayssinet et al., 1997; Upegui-Gonzalez et al., 1998; Varga et al., 1999; Chadwick & Lever, 2000; Terryn & Rouze, 2000; Ji et al. 2001; Wang et al., 2001; De Backer et al., 2001, 2002, 2003; Ji et al., 2002; Martinez et al., 2002; Yin & Ji, 2002; McCaffrey et al., 2003).

SiRNAs inhibit translation through cleavage of their targets, but the mechanism of action of antisense agents is not completely understood. There is evidence that translation inhibition by antisense agents is not always the result of lowering the levels of target mRNA (Probst and Skutella, 1996). In fact, many efficient antisense oligonucleotides function either via steric blocking of the translation machinery (including at regulatory protein binding sites) or by inducing a conformational change in the target RNA, rather than by RNase H-mediated cleavage of the target (Stein, 2000; Toulme, 2001; Braasch and Corey, 2002). These noncleaving modes of action enable the use of antisense techniques in applications for which the elimination or cleavage of mRNA targets would be undesirable, such as regulation (or redirection) of alternative splicing (Volloch et al., 1991a, b; Taylor et al., 1999; Zeng et al., 1999; Gong et al., 2000; Sierakowska et al., 2000; Mercatante et al., 2001) or intracellular imagining of gene expression (Paillasson et al., 1997; Pan et al., 1998; Politz et al., 1998; Tavitian et al., 1998; Chartrand et al., 2000; Molenaar et al., 2001; Pederson, 2001; Dirks et al., 2003). Thus, the ASGS methods provide viable complementary approaches to RNAi technologies (Lavery and King, 2003; Scherer and Rossi, 2003).

The need for antisense agents that are more potent, selective, robust and reliable is widely recognized, which has led to the development of new agents with higher binding affinity and selectivity, as well as schemes for their selection using sequence libraries (Sohail & Southern, 2000). It appears that the affinity of antisense sequences for their target sites (Walton et al., 1999) and the kinetics of hybridization (Patzel & Sczakiel, 2000) are the most important factors in the efficacy of antisense agents. Recently, several new nucleic acid derivatives, including N3′-P5′ and morpholino phosphorodiamidate, 2′-O-methoxyethyl and 2′-fluoroarabino-nucleic acid, have been shown bind strongly to target RNA without resulting in cleavage of the target (Toulme, 2001; Braasch & Corey, 2002; Heasman, 2002; Kurreck, 2003). However, all of these nucleic acid molecules are artificial and, therefore, cannot be expressed by transcription, which is a very good way of providing the high intracellular concentrations that are required for efficient translation inhibition. DNA and RNA may have less stability in vivo than their chemically modified derivatives, but they both can be efficiently and systemically expressed in situ from appropriate PCR templates, plasmids and viral vectors. RNA has certain advantages over DNA—it can be more efficiently expressed in cells than DNA (e.g., using the U6 or H1 pol III promoters) (Noonberg et al., 1994), and RNA-RNA duplexes are more stable than DNA-RNA hybrids (Beckmann & Daniel, 1974; Roberts & Crothers, 1992; Lesnik & Freier, 1995; Landgraf et al., 1996; Wu et al., 2002). When antisense RNAs anneal to complementary sequences of the target transcript, they may affect RNA stability or translation directly, or cause the target transcript to be retained in the nucleus, or stimulate an RNA interference and/or PKR-interferon response (Kumar & Carmichael, 1998). RNA-RNA duplexes, although they are not substrates for RNase H, can be degraded (with some constraints on the antisense sequences) by Dicer, RNase L and RNase P ribonucleases (Kumar & Carmichael, 1998; Terryn & Rouze, 2000; Di Serio et al., 2001; Martinez et al., 2002; Tijsterman et al., 2002; Holen et al., 2003; Pulukkunat et al., 2003; Raj & Liu, 2003). Although at one time the concept of using RNA as a gene inhibitor seemed unlikely due to concerns about the RNA stability in cells, there is now clear evidence of the potential efficacy of RNA-based drugs (Sullenger & Gilboa, 2002).

The development of circularizable nucleic acids is a significant advance in the fields of nucleic acid-based therapeutics and hybridization probe diagnostics. Because of the helical nature of nucleic acid duplexes, circularized probes are wound around the target strand, topologically connecting the two polynucleotides (FIG. 1, bottom). Circularizable oligonucleotides are expected to provide higher efficacy of gene-expression inhibition than linear ones because of the superior stability of the topologically linked nucleic acid complexes versus nucleic acids bound by simple hybridization. Gryaznov and Lloyd (1995) pioneered the design of so-called DNA “clamps,” which can be circularized around the target using a chemical reaction between non-nucleotide reactive groups at the ends of the circularizable nucleic acid. “Padlock” probes (also known as C-probes or CLiPs), which circularize upon treatment with DNA ligase when their ends are brought together by hybridization to adjacent sites on a target DNA or RNA sequence (FIG. 1A), were introduced by Landegren and co-workers (Nilsson et al., 1994; Landegren et al., 1996; Lizardi et al., 1998; Escude et al., 1999; Zhang et al., 1998; Thomas et al., 1999; Antson et al., 2000; Nilsson et al., 2000; Baner et al., 2001; Christian et al., 2001; Hafner et al., 2001; Myer & Day, 2001; Nilsson et al., 2001; Qi et al., 2001; Kuhn et al., 2002; Hardenbol et al., 2003). Padlock probes combine the ability to discriminate point mutations with optional amplification by rolling circle amplification (RCA). It should be noted that DNA clamps, mentioned above, cannot be amplified by RCA because of the unnatural internucleotide link where the ends were joined.

The specificity and efficacy of padlock probes depends on the fidelity and efficiency of a DNA ligase enzyme for the ligation of substrate sequences on different templates. However, DNA ligase does not always discriminate against mismatched sequences. Both terminal and internal mismatches, which frequently occur upon hybridization (especially in GC-rich regions), can be joined by DNA ligase (Goffin et al., 1987; Wu & Wallace, 1989a, b; Luo et al., 1996; Pritchard & Southern, 1997; James et al., 1998). In vitro selection experiments of sequences that can be ligated most efficiently by T4 DNA ligase (using substrate sequences with randomized nucleotides) showed that many of the selected sequences had one or more mismatches even at the ligation junction (Harada & Orgel, 1993; James et al., 1998; Vlassov et al., 2004). However, it should be noted that the accuracy of DNA ligase could be enhanced under certain conditions (e.g., at elevated temperatures using thermostable DNA ligases, high sodium chloride and low ligase concentrations) (Luo et al., 1996). It is possible to optimize ligation conditions for an individual sequence to enable SNP detection in vitro (Luo et al., 1996), but these conditions will not generally be optimal for other sequences or oligonucleotide libraries. Also, ligation of DNA termini aligned on RNA targets occurs with very low efficiency (Nilsson et al., 2000; Nilsson et al., 2001), thus limiting their use as translation inhibitors.

Padlock probes are typically 70-100 nt in length (Antson et al., 2000) and, therefore, require laborious purification by preparative HPLC and gel-electrophoresis since the specificity and efficacy is absolutely dependent on the purity of the material. The proportion of imperfect synthetic oligonucleotide sequences increases with length as a result of incomplete nucleotide deprotection, depurination followed by strand scission during deprotection, premature termination of synthesis, and dephosphorylation (Kwiatkowski et al., 1996). Padlock probes missing terminal nucleotides or 5′-phosphate cannot be specifically ligated. These shorter or dephosphorylated sequences will also compete for target sequence, thus, reducing the yield of perfect ligations. Alternatively, padlock probes can be prepared by asymmetric PCR (Antson et al., 2000; Myer & Day, 2001). A drawback of this method is that DNA polymerases either tend to add (if they are exonuclease-minus) or to remove nucleotides at the 3′-end of the synthesized strand (if exonuclease-plus) (Antson et al., 2000). In both cases, padlock probe circularization is inhibited due to the ligation requirement for perfect ends. The maximum reported yield of ligatable padlock probe sequences after careful optimization of PCR protocols using exonuclease-minus DNA polymerase is only 60-70% (Antson et al., 2000).

“Lassos™” are an additional class of nucleic acid molecules that can circularize around and form topological links to target molecules (FIG. 1B). In addition to an antisense sequence element, they contain a ribozyme moiety that allows them to excise themselves from primary transcripts (removing all unnecessary sequences and creating exact ends required for circularization) and, following hybridization to polynucleotide targets, self-ligate their ends to create topologically-inked complexes (Johnston et al., 1998, 2003). In contrast to clamps and padlock probes (FIG. 1A), the ends of Lassos are not hybridized to the target (FIG. 1B), and the circularization of Lassos requires neither an external protein ligase nor ends that have reactive non-nucleotide groups. Natural RNA sequences may be used, and the ribozyme usually used to ligate the ends is the hairpin ribozyme (HPR), which has distinctive stem and -loop structural features and is efficient at both cleavage and ligation of RNA. Any one of the interdomain loops 1-3 of the HPR (FIG. 2) can be used for introduction of additional sequences without appreciable perturbation of the catalytically-active structure (Feldstein & Bruening, 1993; Komatsu et al., 1993, 1995; Berzal-Herranz & Burke, 1997; Kisich et al., 1999; Fedor, 2000). By attaching an antisense sequence adjacent to the ribozyme core, the Lasso can hybridize to a target nucleic acid, resulting in intertwining of the two polynucleotides. The ends can then refold into the HPR native structure and undergo self-ligation, creating a link between the polynucleotides that has the strength of covalent bonding. Thus, the ribozyme is used not to cleave the target but to cause a hybridized inhibitor to become topologically linked to it. RNA circularization also makes Lassos resistant to exonucleases.

A previously-described RNA Lasso ATRL (FIG. 3A) was designed to bind to a site in the coding region of mouse tumor necrosis factor alpha (TNFα) mRNA (Johnston et al., 1998, 2003). Synthesis of ATR-1 by transcription of a DNA template using T7 RNA polymerase leads to spontaneous self-processing of the transcript (UP) by the internal HPR, resulting in half- (5′-HP, 3′-HP) and fully-processed (L) linear RNA species. An additional species C, which is the covalently closed, circular form of L (FIG. 4), is also produced by the HPR's ability to ligate the ends of the L form (either in the presence or absence of target). The L and C forms spontaneously interconvert in a dynamic equilibrium. ATRL hybridizes rapidly with its target RNA (FIG. 3B), forming strong complexes that are stable enough to be detected by denaturing PAGE containing 8M Urea (FIGS. 3B-C). Upon increasing the temperature, the complex with linear Lasso dissociated at a lower temperature than the target complex with circular Lasso, showing that circular Lasso complexes are more stable than linear complexes (lanes 3 and 4 in FIG. 3C).

RNA Lassos have been successful in inhibiting gene expression in several model systems (Johnston et al., 2000, 2002; Seyhan et al., 2001). Specifically, it has been shown that the enhanced stability of binding between Lassos and TNFα target can provide better inhibition of protein synthesis than ordinary antisense RNA. The 20-nt TNF target sequence was fused to a luciferase reporter gene and a T7 RNA polymerase promoter was attached upstream to create the cassette T7-TNF-Luc (FIG. 5A), which was then inserted into a pGL3 vector (Promega). Transcription of this cassette produced a T7-TNF-Luc target RNA which was then pre-hybridized with either the ATR1 Lasso or AT, an antisense molecule lacking the self-circularizing ribozyme domain but otherwise identical to ATR1. The complexes were used as templates for translation using a rabbit reticulocyte lysate (Promega). Luciferase activity assays (in six separate experiments) revealed that, for an optimal ATR1/target molar ratio of 30:1, ATR1 provided 98% knockdown of translation, whereas AT was virtually ineffective (FIG. 5 B-C).

To assess Lassos' in vivo efficacy in cultured cells, the RNA Lassos, complexed with cationic lipids, were delivered to a macrophage-like cell line, RAW264.7, testing for their ability to inhibit TNFα secretion following stimulation of the cells with lipopolysaccharide (LPS). Different Lasso constructs (including ATR1 and four others) targeted to different sites on the TNFα mRNA (including both 5′-UTR and coding sequences) were tested. A Lasso construct (M101), lacking any sequence complementary to TNFα mRNA, was used as a negative control. The Lassos had an inhibitory effect that was evident for at least 24 hours after LPS stimulation, reducing TNF secretion up to 90% at a level (10 μg) that caused no nonspecific toxicity (IC₅₀ of 46 nM). No inhibition of secretion was observed with M101 at similar levels. In other experiments in which multimers of ALR229 were delivered through a cytoplasmically replicating viral vector based on Semliki Forest virus (SFV), about 95% inhibition was seen (Johnston et al., 1998).

Not all Lassos have been found to be effective, presumably because of their differing abilities to access their target mRNA sites and to circularize around the target.

In general, the efficacy of antisense-based gene inhibitors is dependent on both position and sequence of the target sites, but this efficacy does not always correlate with RNA target site accessibility (Far & Sczakiel, 2003). The use of antisense agents is complicated by the lack of convenient, reliable methods for selecting the most sensitive target sequences. In most cases, potential target sites must be screened individually to find one that allows efficient knockdown of gene expression. But the ‘trial-and-error’ methods for identifying accessible sites are laborious and expensive. Consequently, attempts have been made to select superior sites through computer prediction and in vitro combinatorial approaches (Stull et al., 1996; Bruce & Lima, 1997; Lima et al., 1997; Matveeva et al., 1997; Milner et al., 1997; Ho et al., 1996, 1998; Patzel & Sczakiel, 2000; Lloyd et al., 2001; Sohail & Southern, 2000; Wrzesinski et al., 2000; Allawi et al., 2001; Pan et al., 2001; Scherr et al., 2001; Sczakiel & Far, 2002; Yang et al., 2003). However, there is frequently little correlation between sequences identified as accessible through these procedures and sequences that are truly active as target sites in living cells (Laptev et al, 1994; Yu et al., 1998; Sczakiel & Far, 2002). This incongruity may reflect the different folding of the RNAs within the microenvironments of the living cell versus in cell-free media or as a result of their interactions with RNA-binding proteins in cells. Because there is a poor track record of these methods in predicting accessible regions in vivo, some effort has been made to perform target site selection using randomized antisense sequence libraries (Lieber & Strauss, 1995; Kramer et al., 1997; Kruger et al., 2000; Kawasaki & Taira, 2002). Thus, there is a need for better methods of selecting target sites for nucleic acid-based targeting agents.

There is also a need for improved Lassos that exhibit greater effectiveness in forming a topological linkage with a target and control over sequence specificity in target binding.

### BRIEF SUMMARY OF THE INVENTION

The invention provides a novel class of allosterically-regulated polynucleotide molecules (also termed “Lassos”) that have advantages over currently-existing nucleotide binding agents which may be used, for example, for gene target imaging, detection, and inhibition. These molecules can undergo target-dependent self-circularization to become became topologically linked with nucleic acid targets. Previously-described Lassos contain a non-allosterically-regulated hairpin ribozyme (HPR) catalytic domain that can spontaneously adopt either a linear or circular conformation. Allosteric regulation in the present invention is achieved by converting a target-binding antisense sequence into a “sensor” sequence that binds to a regulatory nucleic acid sequence, serving to either block catalysis by a catalytic domain or lock the Lasso into an open conformation in the absence of target binding, thus preventing self-circularization of the Lasso prior to hybridization with the target. The sensor-antisense sequence is designed so that it has higher affinity to the complementary target sequence, which serves as a catalytic “effector,” than to the regulatory element. Upon binding of the sensor-antisense sequence to the to the target-effector sequence, a conformational (structural) rearrangement occurs, allowing circularization of the Lasso around the target, via formation of either a covalent bond (ligation) between two nucleotide residues of the Lasso or strong non-covalent bonds such as H-bonds, stacking interactions and coordination bonds involving metal ions (or a combination of two or more of these types of bonds and interactions).

This scheme of allosteric regulation is similar to the ‘TRAP’-like mechanism previously described for the cleavage reaction catalyzed by the hammerhead ribozyme (Porta & Lizardi, 1995; George et al., 1998; Burke et al., 2002). However, such a scheme has not been previously described to regulate cleavage and ligation activity of hairpin ribozyme. Moreover, the present invention is unique in using an allosterically regulated ribozyme for circularization of a polynucleotide around the target.

An ideal regulatory element must be sufficiently competitive to block both circularization of the Lasso and non-specific hybridization to the target, but not so competitive as to hinder formation of perfectly matched duplex between Lasso antisense and sense target sequences. In addition to the allosteric regulation, such competition may significantly decrease potential Lasso off-target binding. In other words, the regulatory sequence could function as a “stringency element,” increasing sequence specificity of target recognition and binding via “displacement hybridization” (Roberts & Crothers, 1991; Hertel et al., 1998; Bonnet et al., 1999; Ohmichi & Kool, 2000). A recent report of the high selectivity of oligonucleotide probes containing self-complementary elements to single-nucleotide mismatches or deletions suggest that even single-nucleotide mutation (SNP) discrimination is possible (Li et al., 2002).

Antisense sequences in Lasso constructs may be either rationally designed based on available experimental data or selected by an appropriately modified SELEX technique using a randomized Lasso library, as described in greater detail below. Such libraries may contain either fully random or the directed antisense libraries. A selected antisense sequence may be used to rationally design regulatory elements. In addition, other parts of a Lasso, including catalytic and non-catalytic sequences, or even both sensor and antisense sequences simultaneously, may be at least partially randomized to select/optimize the allosteric regulation and circularization activity. As discussed below, we have prepared allosterically-regulated Lassos against a model target, muTNFα mRNA, and demonstrated both their specific binding to the target and target-dependent circularization. Circularization provides very strong binding to a nucleic acid target, as well as increased resistance to exonucleases.

Circularized and topologically linked Lassos may be selectively amplified even at a trace amount by rolling circle amplification (RCA) or/and RT-PCR as described herein. Such amplification may be used for both detection of specific RNA targets and the selection of optimal Lasso constructs that bind these targets and circularize around target efficiently. Such strong and specific binding may be used for detection and/or inhibition of functions of a target molecule. As described in greater detail below, we have developed and demonstrated the feasibility of such a selection scheme.

To select for Lassos that can circularize efficiently around a target, randomized, Lasso libraries were prepared and exposed to an mRNA target. The resulting strong (stable) Lasso-target complexes were isolated, and the circularized Lasso molecules were selectively amplified by RT-PCR as shown in FIG. 6. The resulting PCR products were used as templates for transcription of RNA Lassos for another round of target binding and selection as shown in FIG. 7. After several rounds, the DNA templates are cloned and sequenced. The selected RNA Lasso sequences were re-synthesized and tested for their ability to tightly and specifically bind the target in vitro and inhibit translation both in vitro extracts and in cultured cells. After rational optimization of selected sequences for optimal sequence specificity (if necessary), the Lasso constructs can be used, for example, for target validation and gene function analysis, antiviral, antibacterial, and gene-therapy drugs.

Optimized Lassos can also be used as hybridization probes (e.g., for Northern blots, in situ hybridization, and microarrays), with utility in the fields of genomics, biodefense, forensics, microbiology, virology and oncology. Topologically linked Lasso-target complexes provide greatly increased binding strength while the sensor element responsible for allosteric regulation also provides higher sequence specificity compared to ordinary cRNA probes since it competes effectively for binding of the antisense sequence with mismatched targets, but is efficiently competed by matched targets. Rolling circle amplification (RCA) of the circularized probe by reverse transcription alone or reinforced by PCR provides very sensitive detection. Topologically linked probes can survive in a complex with circular or immobilized targets at high stringency. Because both washing and the RCA steps allow only probes that have undergone target-specific circularization to be detected, there is a significant enhancement in the signal to noise ratio. Alternative methods of detection that can be used include, for example radioactive, fluorescent, hapten, or enzymatic labels, or binding pairs such as biotin-avidin or streptavidin, which may be directly or indirectly incorporated into the probes during chemical or enzymatic synthesis or by post-synthetic modification. The selection approaches described above could rapidly provide probes which are capable of fast, specific hybridization to accessible target sites, target-dependent circularization, and topological linkage to a target.

In one aspect, the invention provides an allosterically-regulatable polynucleotide which is capable of specifically binding to a target nucleic acid molecule and circularizing around the target, forming a topological linkage.

In one embodiment, polynucleotides of the invention comprise a target binding sequence which is at least partially complementary to and capable of binding to a sequence of the target, and a catalytic domain which is capable of a catalytic activity that is inhibited or prevented from occurring in the absence of binding of the polynucleotide target binding sequence to the target. In the presence of binding of the target binding sequence to the target, the catalytic activity of the catalytic domain catalyzes circularization of the polynucleotide around the target, forming a topological linkage of the polynucleotide to the target. In some embodiments, the catalytic activity is a ligase activity and the catalytic domain catalyzes ligation between two nucleotide residues of the allosterically-regulatable polynucleotide. In one embodiment, the ligase activity catalyzes ligation between 5′ and 3′ ends of the polynucleotide. In another embodiment, the ligase activity catalyzes ligation between the 5′ end of the polynucleotide and a 2′ hydroxyl group of an internal nucleotide of the polynucleotide, thereby forming a “lariat” shaped structure around the polynucleotide. A lariat structure has a free, unligated end to which a detectable label may be optionally attached.

The catalytic domain of polynucleotides of the invention may include RNA or DNA residues or both, or analogs and/or modified forms of these nucleotides thereof. In some embodiments, the catalytic domain comprises, consists of, or consists essentially of RNA residues or analogs and/or modified forms thereof (e.g., the catalytic domain of a ribozyme, for example, comprising, consisting of, or consisting essentially of the catalytic domain of a hairpin ribozyme). In some embodiments, the catalytic domain comprises, consists of, or consists essentially of DNA residues or analogs and/or modified forms thereof (e.g., the catalytic domain of a deoxyribozyme).

In some embodiments, catalytic activity of the catalytic domain is inhibited by a regulatory sequence that is at least partially complementary to and binds to at least a portion of the target binding sequence, rendering the catalytic activity dependent on binding of the polynucleotide to the target. Circularization of the polynucleotide and topological linkage of the polynucleotide to the target are prevented in the absence of target binding, and are permitted upon binding of the polynucleotide to the target. In some embodiments, the conformation of the polynucleotide in the presence of the bound regulatory element prevents access of the catalytic domain to the substrate sequences required for circularization of the polynucleotide around the target.

In another embodiment, polynucleotides of the invention comprise a target binding sequence that is at least partially complementary and capable of specification binding to a target sequence, and circularization proceeds via noncovalent interaction between sequences of the polynucleotide, creating a loop or circular domain that encompasses the target binding domain. In this embodiment, a catalytic domain is not required. Allosteric regulation is accomplished by a conformational change in the polynucleotide upon target binding. For example, allosteric regulation may be achieved by alternative folding that partially occludes the target binding sequence in the absence of target binding. Upon binding of the polynucleotide to the target, the alternative interaction is disrupted, allowing base pairing to occur, circularizing the polynucleotide around the target and producing a loop to which the target is topologically linked (FIG. 9B).

The nucleic acid target may include RNA or DNA residues or both, or analogs and/or modified forms of these nucleotides thereof. In some embodiments, the target comprises, consists of, or consists essentially of RNA residues or analogs and/or modified forms thereof (e.g., mRNA). In some embodiments, the target comprises, consists of, or consists essentially of DNA residues or analogs and/or modified forms thereof (e.g., cDNA, genomic DNA). In one embodiment, the target is single stranded. In another embodiment, the target is double-stranded, and targeting may be via formation of a triplex or D-loop complex between the target and the polynucleotide of the invention.

Allosterically-regulatable polynucleotides of the invention may be prepared by chemical synthesis, or by in vitro or in vivo transcription from an expression vector. In one embodiment, the polynucleotides are transcribed in vitro using RNA polymerase, for example, phages T7, SP6, or T3. In another embodiment, polynucleotides are transcribed in the nucleus of a host cell, for example, by RNA polymerase II or III. The invention also provides allosterically-regulatable polynucleotides prepared by any of the methods described herein.

The synthetic or in vitro transcribed allosterically-regulatable polynucleotide can be either delivered to cellular targets either directly in liposomal complexes or they can be expressed in situ using plasmids or viral vectors. In the case of expression of allosterically-regulatable polynucleotide constructs in the nucleus by Pol II RNA polymerase (using an appropriate expression vector), the suppression of 3′ end processing of the polynculeotide could provide poly(A)-mediated, enhanced export of the polynucleotide to the cytoplasm. Pol 11-mediated polyadenylation may also provide additional nuclease resistance and help attract proteins with helicase activity that may help in binding to a structured mRNA target site (Kawasaki et al., 2002).

In another aspect, the invention provides a complex comprising an allosterically-regulatable polynucleotide as described above circularized around and topologically linked to a nucleic acid target molecule.

In another aspect, the invention provides methods for circularizing a polynucleotide around a target nucleic acid molecule, forming a topological linkage with the target. Such methods comprise contacting a target nucleic acid molecule with an allosterically-regulatable polynucleotide as described above, wherein binding of the polynucleotide to the target, via the target binding sequence of the polynucleotide, either alleviates inhibition of the catalytic activity of the polynucleotide or unblocks sequences required for circularization, thereby allowing topological linkage via circularization of the polynucleotide around the target to occur. The invention also provides methods for reducing the efficiency of transcription and/or translation from a target nucleic acid, comprising circularizing and topologically linking an allosterically-regulatable polynucleotide as described above to the target according to the methods described herein.

In another aspect, the invention provides methods for detecting the presence or absence of a target nucleic acid molecule. Such methods comprise contacting a composition suspected of containing the target with an allosterically-regulatable polynucleotide as described above and detecting circularization and complex formation of the polynucleotide with the target, wherein circularization and complex formation is indicative of the presence of the target in the composition, if any. In one embodiment, the target is linked to a solid support, for example, a hybridization membrane. In another embodiment, the allosterically-reulatable regulatable polynucleotide is linked to a solid support. Optionally, a plurality of the polynucleotides may be provided as an array. In some embodiments, detection of the bound and topologically linked polynucleotide is via amplification of the bound polynucleotide, such as, for example, by rolling circle amplification (RCA) as described and exemplified herein. Other suitable amplification procedures are well known in the art, such as, for example, polymerase chain reaction (PCR), RT-PCR, or isothermal amplification methods. The isothermal methods include displacement amplification (Spargo et al., 1996), transcription-mediated amplification (Pastemack et al., 1997), self-sustained sequence replication (3SR) (Mueller et al., 1997), nucleic acid sequence based amplification (NASBA) (Heim et al., 1998), an assay based on the formation of a three-way junction structure (Wharam et al., 2001), ramification amplification (Zhang et al., 2001), loop-mediated amplification (LAMP) (Endo et al., 2004; Nagamine et al., 2002).

In some embodiments, detection is via a detectable label, which may be included on the allosterically-regulatable polynucleotide, the target, or both. Examples of suitable detectable labels are well known in the art, including but not limited to, radioactive, fluorescent, hapten, or enzymatic labels, or labels that comprise members of ligands capable of tight binding, such as biotin-avidin, biotin-streptavidin, antibody-antigen, etc. In other embodiments, detection is via signal amplification methods, including, for example, serial invasive signal amplification reaction (Hall et al., 2000; Olson et al., 2004), branch chain DNA (b-DNA) technology (Wiber, 1997), tyramide signal amplification (TSAD) and catalyzed assisted reported deposition (CARD) (Rapp et al., 1995).

In another aspect, the invention provides a library comprising a plurality of allosterically-regulatable polynucleotides as described above, and methods for preparing such libraries are described and exemplified herein. In various embodiments, libraries of the invention comprise at least one partially randomized sequence in at least one of the target binding sequence, the catalytic domain, and the regulatory sequence.

The invention also provides a method for selection of polynucleotides that are capable of circularizing around and topologically linking to a target nucleic acid molecule. Such methods comprise contacting the target with a plurality of polynucleotides from a library as described above, and amplifying the polynucleotides which become topologically linked to the target. Optionally, multiple rounds of amplification and selection may be performed to increase the specificity of binding of the selected polynucleotides to the target.

In another aspect, the invention provides kits. In one embodiment, the kit comprises an allosterically-regulated polynucleotide as described above. In another embodiment, the kit comprises a library comprising a plurality of allosterically-regulatable polynucleotides as described above.

### BRIEF DESCRIPTION OF THE DRAWINGS

FIG. 1 schematically depicts circularizable nucleic acid agents: Padlock Probes (A), RNA Lasso (B). These agents are linear polynucleotides that can hybridize to target DNA or RNA. Their terminal sequences are joined by either DNA ligase (Padlock Probes, DNA) or self-ligated by the encoded ribozyme (Lasso, RNA). Circularization of linear agents pre-bound to the target results in formation of topologically linked complexes. C. Depiction of topologically linked polynucleotide-target complex showing interwinding of strands.

FIG. 2 schematically depicts the consensus structure of the hairpin ribozyme (HPR). The HPR is derived from sequences in the minus strand of Tobacco ringspot virus satellite RNA (sTRSV). The site-specific RNA cleavage induced by the ribozyme generates fragments having 2′,3′-cyclic phosphate and 5′-OH termini. HPR can efficiently ligate those ends and can exist as linear and circular forms that interconvert. Both cleavage and ligation reactions require Mg²⁺ under physiological conditions. The internal equilibrium between circular and linear forms depends on the relative stability of the cleaved and ligated forms. Loops 1-3 are not essential for the ribozyme activity (Feldstein & Bruening, 1993) and could be deleted or extended (e.g., antisense and regulatory element sequences can be inserted). Loop A represents the template-substrate complex and Loop B represents the catalytic core. Dots represent any nucleotide (A, U, G or C), dashes represent required pairings, V is ‘not U’ (A, C, or G), Y is a pyrimidine (U or C), R is a purine (A or G), B is ‘not A’ (U, C or G), H is ‘not G’ (A, C or U) (Berzal-Herranz & Burke, 1997).

FIG. 3 depicts binding of Lasso ATR1 to TNF RNA target. A. Schematic depiction of complex between the TNF-705 (comprising 280-985 nts in murine TNFα mRNA) and the fully processed ATR1 Lasso (which targets 562-583 nts in TNF target). B. Time course of binding of ATR1 with TNF RNA. ³²P-labeled TNF target was incubated with cold ATR1 Lasso at 37° C. for the time periods indicated above each lane on the gel. Complex formation was carried out in either 50 mM Tris-HCl (pH 8.0), 10 mM MgCl₂, 20% formamide (left) or 50 mM Tris-HCl (pH 8.0), 10 mM MgCl₂ (right). Following complex formation, reactions were quenched with one volume of loading buffer containing 95% formamide, 10 mM EDTA. 3C. Heating-induced dissociation of complexes formed by TNF target RNA with linear and circular Lasso species. ³²P-labeled Lassos were incubated at 37° C. for 2 hrs in buffer containing 50 mM Tris-HCl (pH 8.0), 10 mM MgCl₂ with and without cold TNF target RNA (in excess over Lassos) as indicated. Reactions were quenched as described in B. Samples in Lanes 1 and 2 were not incubated further. Samples in lanes 3, 4, 5, and 6 were additionally incubated for 2 min at 50°, 65°, 80°, and 95° C., respective transferred immediately to ice to prevent re-hybridization of the complexes. Products were analyzed by 6% denaturing PAGE (8M Urea).

FIG. 4 schematically depicts processing of a Lasso hairpin ribozyme.

FIG. 5 shows inhibition of translation in vitro by Lasso RNA (ATR1) in a rabbit reticulocyte lysate. A. Schematic depiction of DNA template for TNF-1 fusion cassette T7-TNF-luc. B. Inhibition of luciferase activity as a result of pre-hybridization of T7-Luc and T7-TNF-luc mRNAs with either control 5S RNA (lacking antisense TNF sequences), AT antisense or ATR1 Lasso at target/agent ratio 1:30. T7-luc is a control mRNA (lacking the TNF binding site) transcribed from the pGL3-Control Vector with inserted T7 polymerase promoter alone. T7-Luc-TNF is mRNA transcribed from the construct shown in A. Increasing amounts of ATR1 or huIL-1(2); C. Effect of increasing amounts of Lassos on translation. ATR1 Lasso was added to a mixture of TNF/Luc and pro-IL-1 mRNAs (lanes 2-6, lane 1 contains only the TNF/Luc RNA). Following a 40 minute incubation with rabbit reticulocyte lysate in the presence of ³⁵S-labeled Met, the translation products were separated on an SDS 12% polyacrylamide gel and visualized by autoradiography. Lanes 3-6 contain 20, 40, 80 and 160 fold molar excess of ATR1 Lasso with respect to target.

FIG. 6 depicts the sequence and structure of members of a Lasso library with randomized antisense segments (depicted as N). A. Unprocessed Lasso. The position of a primer for selectively extending the circularized (but not linear) Lassos by RT-RCA is indicated. N represents any nucleotide (A, G, C and U). B. Self-processed circular Lassos bound to the complementary sites in target mRNA. Primers for amplifying a RT-RCA product and converting it into a transcription template are indicated.

FIG. 7 schematically depicts a selection scheme for Lasso species that circularize around a target mRNA. A. For each cycle of selection, the Lasso library is incubated with target RNA. Lasso-target complexes are then isolated on denaturing polyacrylamide gel and circular Lassos are selectively amplified by RCA-RT-PCR. B. Lasso species isolated by the procedure depicted in A are reverse transcribed by reverse transcriptase (e.g., Invitrogen) using a primer complementary to the defined 5′-end of all Lassos such that only circular Lassos are extended by rolling circle amplification (RCA), yielding single-stranded DNA multimers of the Lasso sequence. The RCA products are further amplified by PCR to generate a template that then can be used for in vitro transcription. Two additional primers (PCR primer 2 and PCR primer 3) are used to amplify the monomer Lasso sequence, restore the flanking Lasso sequences, and add a T7 promoter at the Lasso 5′ end so that the resulting DNA template can be transcribed in vitro. Since this PCR reaction may yield multiple products, the DNA fragment corresponding to the monomer Lasso sequence may be gel-purified.

FIG. 8 schematically depicts a pool of unprocessed ALR229-SN Lassos and specific sequences ALR229-5, 229-6, 229-7, 229-8, 229-9 and 229-10 which differ in the length of the regulatory element, respectively having 5, 6, 7, 8, 9 and 10 nucleotides complementary to the Lasso's antisense domain. The regulatory sequence includes 5 nt corresponding to the sequence immediately adjacent to the ribozyme cleavage/ligation site. ALR229-5N is a library of Lassos having all four nucleotides (A, G, C and U) at each of the N positions.

FIG. 9 shows examples of target-dependent ciruclarization through covalent (A) and noncovalent (B) circularization. A. Self-processing of Lasso ALR229-8 and binding of the Lasso to the TNF target. The unprocessed pre-Lasso-undergoes a self-cleavage at the 5′ end. The self-cleavage of the 3′ end is inhibited by an intramolecular base-pairing of the 8-nt long sensor element with the Lasso's antisense domain. The sensor sequence includes a 5 nt HPR substrate sequence, which is immediately adjacent to the ribozyme cleavage/ligation site. Upon binding to the target, this 5-nt substrate sequence is released, allowing 3′ end cleavage of the Lasso. The filly processed Lasso, bound to the target, can then undergo circularization. B. Target-dependent circularization without the presence of a ribozyme. In this case, in the absence of target, the Lasso adopts an “open” conformation. Upon binding of the target, the internal pairing of the open conformation is disrupted and the ends hybridize, creating a circular domain encompassing the target binding sequence, thereby circularizing the polynucleotide around the target. In this example, the preferred relative stability of the three base-paired regions is [end-pairing]<[open Lasso pairing]<[target-Lasso pairing].

FIG. 10 shows self-processing of ³²P-internally-labeled allosterically-regulated Lassos and the effect of formamide. Each of the Lassos was incubated in either 50 mM Tris-HCl, pH 8, 10 MM MgCl₂ (−lanes) or 50 mM Tris-HCl, 10 mM MgCl₂, 20% formamide (vol/vol) (+lanes) for 120 minutes at 37° C. Reactions were quenched as described in FIG. 3B. The samples were electrophoresed through 6% polyacrylamide containing 8M urea and 0.5× TBE. C, circular Lasso, UP, unprocessed Lasso, HP, half processed Lasso, L, fully processed linear Lasso.

FIG. 11 shows binding of internally ³²P-labeled allosterically-regulated Lassos to target RNA in 50 mM Tris-Cl, pH 8, 10 mM MgCl₂, 20% formamide. Lassos as described in FIG. 8 were incubated in for 120 minutes at 37° C. either alone (−) or with 1.4 mM target RNA (in excess over Lassos) (+). Reactions were quenched and analyzed as described in FIG. 10.

FIG. 12 shows binding of Lassos ALR229-5 through 10 with TNF2 (709 nt) and TNF-20 (20-nt) target RNAs with target-dependent self-processing and complex formation. Trace amounts of the internally ³²P-labeled Lassos were incubated in 10 mM MgCl₂/50 mM Tris-Cl (pH 8) for a total of 120 minutes at 37° C., either alone (lanes 1) or with non-radioactive 0.4 μM TNF-20 (lanes 2) or 0.4 μM TNF2 (lanes 3-5). Lanes 4 are the same as lanes 3 but chased with a 14-fold excess of 20-nt competitor antisense RNA, anti-TNF-20 over TNF2. Lane 5 is the same as lane 3 but chased with 7-fold excess of competitor sense TNF-20 (20-nt) over TNF2. Samples were analyzed by 6% denaturing PAGE. Anti-TNF-20 is identical to the antisense sequence incorporated-into the Lassos. TNF-20 corresponds to the sequence of TNF-α mRNA targeted by these Lassos. Abbreviations: S is PAGE start; LLT are Lasso complexes with the long target (TNF2); LST are Lasso complexes with the short target (TNF-20); CL are the circular forms of fully-processed Lassos; UPL are unprocessed pre-Lasso transcripts; 5PL are 5′-end semi-processed pre-Lassos; L are fully-processed (at both 5′- and 3′-ends), linear Lasso.

FIG. 13 shows an analysis of target-dependent circularization of Lassos 229-5, -6, -7, -8, -9, and -10. ³²P-internally-labeled allosterically-regulatable Lassos were incubated with target RNA as described in FIG. 11. Reactions were quenched as described in FIG. 10. Half of the Lasso-target complex sample was heated at 90° C. for two minutes followed by immediate transfer to ice. The samples were then loaded onto a 6% PAGE gel containing 8M urea and 0.5× TBE buffer.

FIG. 14 schematically depicts sequences and secondary structures of allosterically-regulatable Lasso 229-7 and variants differing in the 3′-end sequence.

FIG. 15 shows the effect of the length of the 3′ end sequence on processing and target binding for Lasso 229-7. Internally ³²P-labeled Lassos were incubated as described in FIG. 11 and quenched as described in FIG. 10. Half of the Lasso-target complex sample was heated at 90° C. for two minutes followed by immediate transfer to ice. The samples were then loaded onto a 6% PAGE gel containing 8M urea and 0.5× TBE buffer. C, circular Lasso; UP, unprocessed Lasso; HP, half-processed Lasso; L, fully-processed linear Lasso.

FIG. 16 shows target-dependent circularization and heating-induced dissociation of Lasso 229-7(0). 32p labeled Lasso 229-7(0) was incubated either alone (lane 1) or with non-radioactive target RNA (lane 2). at 37° C. in buffer containing 50 mM Tris-Cl, pH 8, 10 mM MgCl₂, 20% formamide. Following complex formation, reactions were quenched as described in FIG. 10. Aliquots of the Lasso-target complex were further incubated at 50° C., 65° C., 80° C., and 95° C. (lanes 3, 4, 5, and 6, respectively) to induce dissociation and placed immediately on ice to prevent re-hybridization. Samples were loaded on a 6% PAGE/8M urea gel. C, circular Lasso; UP, Unprocessed Lasso; HP, Half-processed Lasso; L, 5′ and 3′ processed linear Lasso; CT, Circular Lasso-Target Complex.

FIG. 17 shows target-dependent circularization and Lasso-target complex formation in a comparison between standard buffer and physiological buffer. Internally ³²P-labeled Lassos were incubated as described in FIG. 11 and quenched as described in FIG. 10. Half of the Lasso-target complex sample was heated at 90° C. for two minutes followed by immediate transfer to ice. The samples were then loaded onto a 6% PAGE gel containing 8M urea and 0.5× TBE. C, circular Lasso; UP, unprocessed Lasso; HP, half-processed Lasso: L, fully-processed linear Lasso.

FIG. 18 schematically depicts allosterically-regulatable Lassos containing an antisense sequence to a target sequence of nucleotides 562-583 of murine TNFα.

FIG. 19 shows target binding and target-dependent circularization for ALR-562 series Lassos as depicted in FIG. 18. For each Lasso in the series, lane 1 shows the Lasso incubated at 37° C. for 120 min. without target, lane 2 shows the Lasso+target TT-280 RNA incubated at 37° C. for 120 min., and lane 3 is the same as lane 2, but incubated for an additional 2 min. at 95° C. and then placed immediately on ice prior to loading. C, circular Lasso; UP, unprocessed Lasso; HP, half-processed Lasso: L, fully-processed linear Lasso.

FIG. 20 depicts an analysis of the interaction of circular and linear targets with allosterically-regulatable Lasso 229-7(0). A. Schematic depiction of the sequence and secondary structure of Lasso 229-7(0) unprocessed (top) and bound to target RNA (bottom). B. Internally ³²P-labeled Lasso 229-7(0) was incubated in buffer containing 50 mM Tris-HCl (pH 7.5), 10 mM MgCl₂, 20% formamide (lanes 1-6) or 50 mM Tris-HCl (pH 7.5), 10 mM EDTA, 20% formamide (lanes 7-12) for 120 min. at 37° C. alone, with linear target RNA, or circular RNA as indicated. After incubation at 37° C., reactions were quenched with loading buffer containing 95% formarnide, 10 mM EDTA. Samples incubated with target RNA were split in half. Half was loaded without further incubation (middle lane of each set of three, i.e. lanes 2, 5, 8, 11) and the other half after incubation at 95° C. for 5 min followed by quenching on ice (right lane of each panel, i.e. lanes 3, 6, 9, 12) and analyzed on 6% denaturing PAGE (8M urea). As controls, Lassos were incubated in buffer without target (lanes 1, 4, 7, and 10).

FIG. 21 shows a gel shift analysis of Lassos 229-5 and 229-7(0) binding to target RNAs containing mismatches to antisense sequence. ³²P-labeled Lassos were incubated in buffer containing 50 mM Tris-HCl (pH 7.5), 10 mM MgCl₂, 20% formamide for 120 min. at 37° C. alone, with wildtype target RNA, or mutant target RNAs as indicated. After incubation at 37° C., reactions were quenched with loading buffer containing 95% formamide, 10 mM EDTA and analyzed on 6% denaturing PAGE (8M urea). The mismatched positions with respect to the 229 antisense sequence as well as the sequences of the mutant target RNAs are indicated on the Lasso secondary structure below each gel.

FIG. 22 shows a scheme for amplification of circularized Lassos by RCA-RT-PCR. A. Structure of Lasso 229-5 that targets the,229-248 region of-TNFα with schematically depicted RT-PCR primers. In the reverse transcription (RT) reaction, primer 1 selectively extends only circular Lassos, yielding single-stranded DNA multimers of the Lasso sequence (rolling circle amplification, RCA). Two additional primers (primer 2 and primer 3) were used to amplify the RCA product by PCR and to restore the T7 promoter sequence at the Lassos' 5′ ends so that the products could be transcribed in vitro. B. Results of RCA-RT-PCR for Lasso 229-5. MW is a 100 bp ladder molecular weight marker. Lane 1 is a negative control with no template and no Taq polymerase added. Lane 2 is a negative control with no template added. Lane 3 is Lasso 229-5 amplified by RCA-RT-PCR. The asterisk (*) marks the expected product of the reaction. C. RCA-RT-PCR was performed on the Lasso complex after gel purification. The PCR reaction was allowed to proceed for fewer cycles (15) of amplification than those in the 22B (25 cycles).

### DETAILED DESCRIPTION

The invention provides novel allosterically-regulatable polynucleotide molecules that have advantages over currently-existing nucleic acid binding agents. Polynucleotides of the invention can be used, for example, to reduce the efficiency of transcription or translation, for detection and imaging of nucleic acid targets, target validation, or gene function analysis, or as antimicrobial (e.g., antiviral, antibacterial) drugs, or for gene therapy. Polynucleotides of the invention may also be used, for example, as hybridization probes (e.g., for Northern or Southern blots, in situ hybridization, microarrays) with utility in the fields of genomics, biodefense, forensics, microbiology, virology, and oncology. A polynucleotide of the invention includes both a target binding sequence which is capable of binding to a sequence of a target nucleic acid molecule and an ability to circularize that is inhibited in the absence of binding of the polynucleotide to the target molecule. Upon binding of the target binding sequence to the target, a structural rearrangement occurs, allowing circularization of the polynucleotide around the target nucleic acid molecule. As used herein, “circularization” encompasses both covalent and noncovalent interactions that create a circular domain.

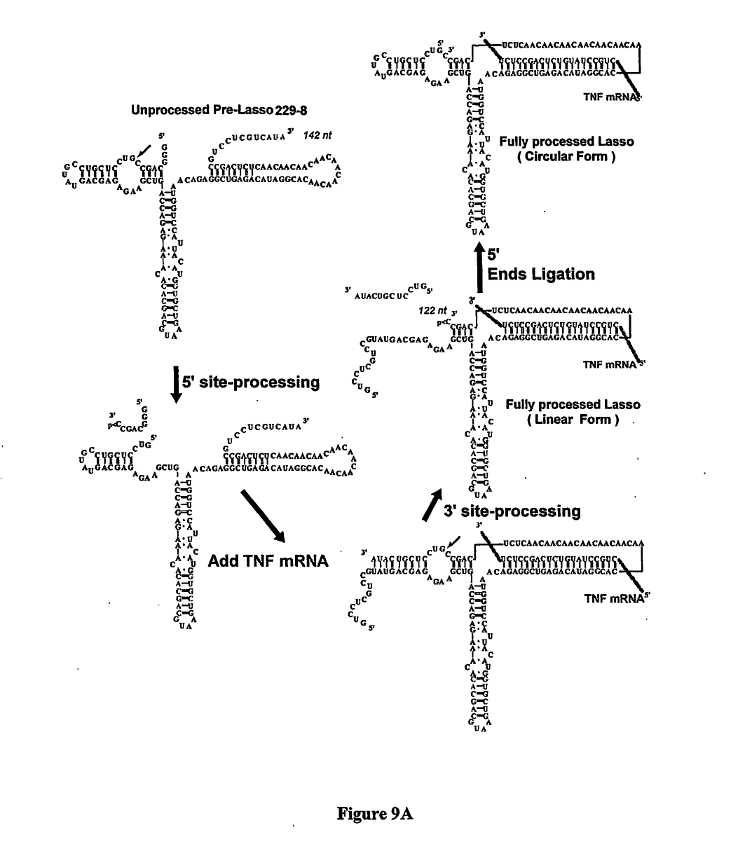

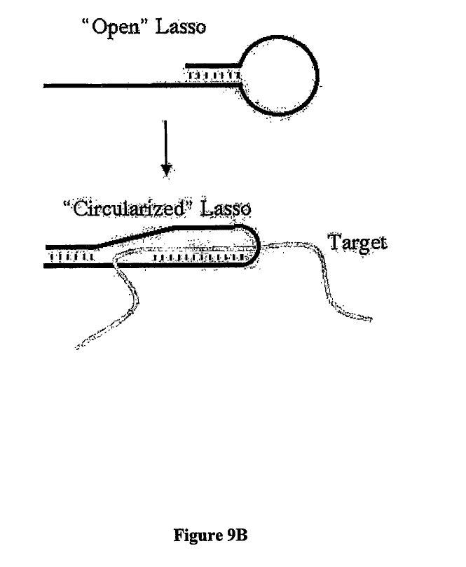

In one embodiment, “circularization” involves noncovalent interactions within the polynucleotide that create a “circular” domain encompassing the target binding region. (FIG. 9B) This circularization is prevented in the absence of bound target by an alternative interaction involving part of the target binding sequence. Upon binding of the target, this alternative interaction is disrupted, inducing a rearrangement that allows circularization around the target.

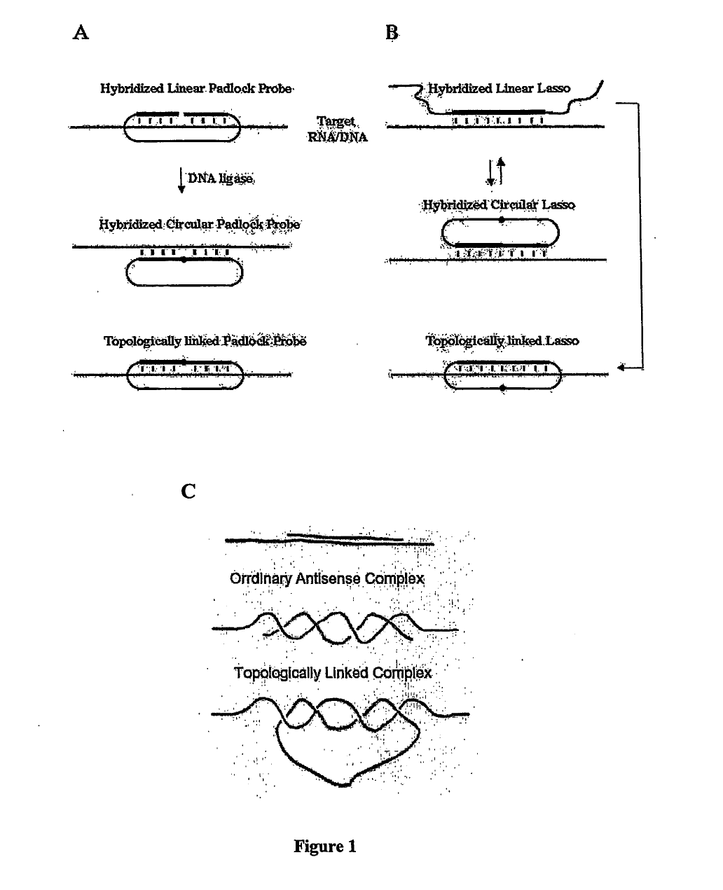

In an another embodiment, the polynucleotide of the invention includes a catalytic domain having an ability to induce circularization that is inhibited in the absence of binding of the polynucleotide to the target molecule. Upon binding of the target binding sequence to the target, a conformational change in the polynucleotide allows catalytic action by the catalytic domain resulting in “circularization” of the polynucleotide around the target. (FIGS. 1B and C)

Previously-described Lassos contain a non-allosterically-regulated hairpin ribozyme (HPR) domain that can spontaneously adopt either a linear or circular conformation. (PCT Application No. WO 99/09045; Australian Patent No. AU756301) In contrast, the polynucleotides of the present invention are allosterically regulated. In the present invention, the efficacy of the Lasso topological linkage to the target and target sequence specificity are enhanced by making the Lasso circularization target-dependent using allosteric regulation.

Allosteric regulation of ribozymes, based on competition between a “sensor” sequence and an external effector sequence supplied by either a synthetic oligonucleotide or a target sequence, has been previously described (Porta & Lizardi, 1995; George et al., 1998; Robertson & Ellington, 2000; Soukup & Breaker, 2000; Warashina et al., 2000; Kazakov, 2001; Burke et al., 2002; Komatsu et al., 2002; Wang et al., 2002; Silverman, 2003). The sensor sequence is designed to be partially complementary to a sequence on or near the ribozyme so as to create in interaction that interferes with the normal functioning of the ribozyme in the absence of the effector. The sensor sequence is designed so that it has higher affinity to the complementary effector sequence than to a functionally important ribozyme sequence. Upon binding of the sensor to the effector, the ribozyme catalytic domain becomes unmasked, and, therefore, active either as a nuclease or ligase or both. Even without extensive rational design, a more than 250-fold rate enhancement in the effector-activated hammerhead ribozyme reaction has been previously observed (Burke et al., 2002; Silverman, 2003). The limiting extent of activation is likely to be proportional to the ratio of relative stabilities of the sensor-effector duplex with the folded core to the sensor-ribozyme complex. It should be possible to further optimize this ratio through rational design or exploiting evolutionary optimization through the in vitro selection (SELEX) (Burke et al., 2002).

General Techniques

The practice of the present invention will employ, unless otherwise indicated, conventional techniques of molecular biology (including recombinant techniques), microbiology, cell biology, and biochemistry, which are within the skill of the art. Such techniques are explained fully in the literature, such as: “Molecular Cloning: A Laboratory Manual,” vol. 1-3, third edition (Sambrook et al., 2001); “Oligonucleotide Synthesis” (M. J. Gait, ed., 1984); “Methods in Enzymology” (Academic Press, Inc.); “Current Protocols in Molecular Biology” (F. M. Ausubel et al., eds., 1987); “PCR Cloning Protocols,” (Yuan and Janes, eds., 2002, Humana Press).

Polynucleotides of the Invention

Polynucleotides of the invention, including those termed “Lassos” herein, specifically bind to a target nucleic acid molecule and circularize around the target. Circularization is dependent on binding of the polynucleotide to a sequence of the target. The polynucleotide contains both a target binding sequence and a means of creating a circular domain that encompasses the target binding sequence. In some embodiments, the means of circularization involves action by a nucleic acid catalytic domain. The catalytic domain is unable to cause circularization in the absence of binding of the target binding sequence to the target. Upon binding of the polynucleotide to the target, the circularization can proceed. Catalytic activity of the catalytic domain serves to circularize the polynucleotide around the target, forming a topological linkage of the polynucleotide with the target. In other embodiments, the means of “circularization” includes formation of noncovalent interactions that create a circular domain around the target, forming a “topological linkage” of the polynucleotide to the target.

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Any type of modified nucleotide or nucleotide analog may be used, so long as the polynucleotide retains the desired functionality under conditions of use, including modifications that increase nuclease resistance (e.g., deoxy, 2′-O—Me, phosphorothioates, etc.). Labels may also be incorporated for purposes of detection or capture, for example, radioactive or nonradioactive labels or anchors, e.g., biotin. The-term polynucleotide also includes peptide nucleic acids (PNA). Polynucleotides may be naturally occurring or non-naturally occurring. The terms “polynucleotide” and “nucleic acid” and “oligonucleotide” as used herein are used interchangeably. Polynucleotides of the invention may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions. The terms “polynucleotide” and “nucleic acid” and “oligonucleotide” as used herein are used interchangeably.

Allosterically-regulatable polynucleotides of the invention include a target binding sequence and may include a catalytic domain. As used herein, “target binding sequence” or “antisense sequence” refers to a sequence that is at least partially complementary and capable of binding to a sequence of a target nucleic acid. In some embodiments, the target binding sequence is about 10 to about 30, often about 20, base pairs in length. The target binding sequence may be fully complementary to the target sequence, or there may be one or more mismatches between the binding sequence and the target, so long as binding is sequence specific and tight enough to disrupt alternative interactions that prevent circularization in the absence of target binding. The target binding sequence generally comprises a sequence that is at least “substantially complementary” to a sequence of the target molecule, meaning a sequence that is sufficiently complementary to allow hybridization therebetween via normal base pair binding. Substantially complementary sequences may be fully complementary or may have one or more mismatch(es). Either or both of the target binding sequence and the target may comprise DNA, RNA, or both, and/or analogs or modified forms thereof, and/or modified internucleotide linkages.

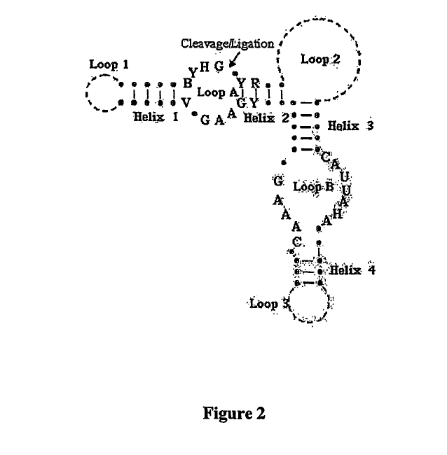

As used herein, “catalytic domain” refers to a nucleic acid sequence that is capable of catalyzing a reaction, for example, ligation between nucleotides or cleavage and subsequent ligation of a nucleic acid sequence. In one embodiment, the catalytic domain is capable of catalyzing a ligation reaction between 5′ and 3′ ends of a polynucleotide molecule of the invention to circularize and topologically link the polynucleotide to the target. An example of such a catalytic domain is the catalytic domain of the hairpin ribozyme, as shown in FIG. 2. In another embodiment, the catalytic domain is capable of catalyzing a ligation reaction between the 2′ hydroxyl group of an internal nucleotide and the 5′ end of a polynucleotide of the invention to form a “lariat” structure when the polynucleotide is circularized around and topologically linked to the target. (For examples of nucleic acid catalyzed 2′-5′ ligase activities, see Prior et al. (2004) Nucleic Acids Res. 32(3):1075-82; Flynn-Charlebois et al. (2003) J. Am. Chem. Soc. 125(18):5346-50; Flynn-Charlebois et al. (2003) J. Am. Chem. Soc. 125(9):2444-54.) The catalytic domain may comprise, consist of, or consist essentially of DNA (for example, the catalytic domain of a deoxyribozyme), RNA (for example, the catalytic domain of a ribozyme), or both DNA and RNA, and/or analogs or modified forms thereof, and/or modified internucleotide linkages, so long as the catalytic activity is sufficient to facilitate circularization of the polynucleotide around the target and topological linkage thereto. (For examples of RNA/DNA chimeric catalytic domains, see Perrault et al. (1990) Nature 344(6266):565-7; Taylor et al. (1992) Nucleic Acids Res. 29(17):4559-65; Shimayama et al. (1992) Nucleic Aids Symp. Ser. 27:17-18; Chowrira et al. (1993) J. Biol. Chem. 268(26):19458-62; Kong et al. (2002) Biochem Biophys Res Comm 292(4):1111-5.) Allosterically-regulatable polynucleotides of the invention generally comprise all of the nucleotide sequences required to form a complete catalytically active catalytic domain. However, in some embodiments, part of the catalytic domain is supplied by sequences of the target. In embodiments where the polynucleotide of the invention contains a catalytic domain, that domain is unable to induce topological linkage of the polynucleotide around the target in the absence of binding of the target binding sequence to the target.

The “target,” “target sequence,” or “target nucleic acid” as used herein is a polynucleotide comprising a sequence of interest. The target may comprise DNA, RNA, or both, and/or analogs or modified forms thereof, and/or modified internucleotide linkages. In some embodiments, the target is mRNA, genomic DNA, cDNA, cRNA, viral RNA, ribosomal RNA, non-coding RNA, a viral RNA-DNA replication intermediate, or an RNA-protein complex. Allosterically-regulatable polynucleotides of the invention become topologically linked to the target by one or more of a variety of mechanisms described herein, regardless of the structure of the target nucleic acid. The target nucleic acid may be linear, circular, or may comprise both linear and circular portion(s), or may take any other form that allows topological linkage of a polynucleotide of the invention thereto.

As used herein, “topological linkage” refers to intertwining of a circularized polynucleotide of the invention with the target nucleic acid molecule (see FIG. 1C), The “linkage number” is determined largely by the length of the pairing interaction and consequently the number of helical turns by which the two molecules are interwound. The topological linkage often serves to make the binding between the target binding sequence and the target resistant to dissociation promoted by helicases, ribosomes or modifying enzymes and, in turn, imparts improved translation or transcription regulatory properties or improved detection of the target. A “topologically linked” polynucleotide herein refers to a polynucleotide that is circularized around the target molecule. It is generally difficult to displace a polynucleotide that is circularized around a target nucleic acid. Unless an endonucleolytic cleavage event occurs in the circular molecule, hydrogen bonds between the two molecules would have to be simultaneously broken, and the target would have to thread its way out of the circle, which would be expected to be kinetically very slow, especially in the case of mRNA targets having significant secondary structure.

Topological linkage of a polynucleotide of the invention is allosterically regulatable, with circularization dependent on target binding. Circularization is blocked in the absence of target binding. In one embodiment, circularization is inhibited by a “regulatory” (also termed “inhibitory” or “inhibitor” herein) nucleic acid which binds to at least a portion of the target binding sequence, thereby preventing circularization of the polynucleotide when it is not bound to the target. In one embodiment, the regulatory sequence is a sequence of the allosterically-regulatable polynucleotide, either internal to the polynucleotide or at one or both of the ends. In another embodiment, the regulatory sequence is on a different nucleic acid than the allosterically-regulatable polynucleotide. In one embodiment, the regulatory nucleic acid sequence comprises a sequence that is at least partially complementary, often substantially complementary, sometimes fully complementary to the target binding sequence. The regulatory element may include mismatches and still maintain high fidelity of binding to the intended target. The regulatory element-target binding sequence binding needs only to be strong enough to block circularization in the absence of binding of the target binding sequence to sequences of the target nucleic acid. Binding between the regulatory sequence and the target binding sequence improves specificity of binding of the polynucleotide to the target through competition between the regulatory sequence and the sequence of the target to which the target binding sequence binds. In embodiments in which circularization is dependent on catalytic activity, binding of the target binding sequence to the target displaces the regulatory sequence, which allows catalytic action by the catalytic domain, resulting in circularization of the polynucleotide around the target.

The invention also provides a complex comprising an allosterically-regulatable polynucleotide as described above circularized around and topologically linked to a target molecule. Often, formamide is included in the reaction mixture for complex formation. In various embodiments, about 5, 10, 15, or 20% formamide is used. Formamide has been reported to provide a stronger correlation between in vitro and in vivo efficacy of ribozymes (Crisell et al., 1993; Kisich et al., 1997; Sullivan et al., 2002). Presence of the complex may reduce efficiency of transcription and/or translation from the target nucleic acid.

Methods for Topologically Linking a Polynucleotide Lasso to a Target Molecule

The invention provides methods for circularizing an allosterically-regulatable polynucleotide molecule as described above around a target molecule, forming a topological linkage. A method of the invention includes contacting a composition containing the target molecule with a polynucleotide that comprises a target binding sequence, wherein binding of the target binding sequence to the target allows circularization and topological linkage of the target to proceed.

In one embodiment, the polynucleotide comprises a target binding sequence and a catalytic domain capable of catalytic action, and binding of the target binding sequence to the target allows catalytic action to proceed, resulting in circularization and topological linkage of the polynucleotide to the target. In the absence of target binding, catalysis does not occur, preventing or significantly reducing circularization and topological linkage to the target. Often, inhibition of catalysis is effected by an regulatory sequence as described above. In one embodiment, the catalytic activity is a ligase activity, causing ligation between the 5′ and 3′ ends of the polynucleotide to form a circular structure around the target. In another embodiment, the catalytic activity is a ligase activity, causing ligation between the 5′ end and a 2′ hydroxyl group of an internal nucleotide residue to form a lariat shaped structure whose circular part is intertwined with the target. 2′-5′ ligation allows potential labeling at the free 3′ end of the polynucleotide, which may be used for detection of topologically linked polynucleotides and targets. As used herein, “ligation” refers to the formation of a phosphodiester bond between a hydroxyl group of one nucleotide and a phosphate group of another nucleotide, e.g., the 3′-OH or 2′-OH of one nucleotide and a 5′-phosphate group of another nucleotide, such that there are no intervening nucleotides between the nucleotides that have been joined by ligation.

In another embodiment, the polynucleotide comprises a target binding sequence and binding of the target binding sequence to the target causes circularization to proceed via structural rearrangement within the polynucleotide that creates a circular domain encompassing the target binding sequence, circularizing the polynucleotide around the target and forming a topological linkage.

The invention also provides methods for reducing the efficiency of transcription and/or translation from a target, or inhibiting or redirecting splicing, comprising topologically linking an allosterically regulated polynucleotide to the target as described above. Transcription and/or translation may be partially reduced or fully eliminated. Reduction of transcription or translation may be detected by methods that are well known in the art including, but not limited to, Northern or Southern blots or RT-PCR for transcription or Western blots or ELISA (enzyme-linked immunorbant assay) for translation. In various embodiments, transcription or translation is reduced at least about 10, 20, 30, 40, 50, 60, 70, 80, or 90%, or is fully eliminated, depending on specific factors such as the accessibility of the target site, the efficiency of binding and regulation of the polynucleotide and other influences, due to formation of a complex comprising a topologically linked polynucleotide of the invention to the target.

In principle, allosterically regulated polynucleotide-target complexes can disable target RNA by three distinct processes: physically blocking its functional sequences, disruption of functionally active structures, and induction of its degradation. Within these broadly defined processes, different mechanisms are possible. These mechanisms include but are not limited to translation arrest, prevention of RNA processing, regulation of alternative splicing or nuclear retention of target transcripts, depending on the design of the allosterically regulatable polynucleotide, the target sequence chosen, or the selection procedure used to identify effective allosterically regulated circularizable polynucleotides from combinatorial libraries. Allosterically regulatable polynucleotides of the invention can be delivered to cellular targets either directly in liposomal complexes or through expression in situ from plasmids or viral vectors.

Methzods for Detecting Presence or Absence of a Target Nucleic Acid Molecule

The invention provides methods for detecting the presence or absence of a target nucleic acid molecule. Methods of detection include contacting a composition suspected of containing a target molecule with an allosterically-regulatable polynucleotide as described above, and detecting circularized polynucleotide topologically linked to the target, wherein presence of the circularized polynucleotide indicates presence of the target molecule in the composition, if any, and absence of the circularized polynucleotide indicates absence of the target molecule.

In one embodiment, the target molecule is associated with or bound to a solid support, e.g., a hybridization membrane, e.g., nitrocellulose or nylon (dot blots, Northern blots, Southern blots), modified glass, silicon or gold surfaces (microarrays), modified magnetic or glass beads (affinity capture).

In another embodiment, the allosterically-regulatable polynucleotide molecule is associated with or bound to a solid support. For example, the polynucleotide may be comprised within an array. “Microarray” and “array,” as used interchangeably herein, comprise a surface with an array, preferably ordered array, of putative binding (e.g., by hybridization) sites for a biochemical sample (target) which often has undetermined characteristics. In a preferred embodiment, a microarray refers to an assembly of distinct allosterically-regulatable polynucleotides as described above immobilized at defined positions on a substrate. Arrays are formed on substrates fabricated with materials such as paper, glass, plastic (e.g., polypropylene, nylon, polystyrene), polyacrylamide, nitrocellulose, silicon, optical fiber or any other suitable solid or semi-solid support, and configured in a planar (e.g., glass plates, silicon chips) or three-dimensional (e.g., gels, pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Naskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Polynucleotides may also be noncovalently immobilized on the substrate by hybridization to anchors, by means of magnetic beads, or in a fluid phase such as in microtiter wells or capillaries.

Detection of topologically linked polynucleotides may be by any of a number of methods that are well known in the art, including, for example, detecting a, label on either the allosterically-regulatable polynucleotide molecule and/or the target nucleic acid molecule. Detectable labels include, for example, radioisotopes (e.g., ³H, ³⁵S, ^{=P,} ³³P, ¹²⁵I, or ¹⁴C), fluorescent dyes (e.g., fluorescein isothiocyanate, Cy3, Cy5, Texas red, rhodamine, green fluorescent protein, and the like), enzymes (e.g., LacZ, horseradish peroxidase, alkaline phosphatase, luciferase), digoxigenin, and colorimetric labels such as colloidal gold or colored glass or plastic (e.g., polystyrene, polypropylene, latex, etc.) beads, and members of binding pairs such as biotin-avidin, biotin-strepavidin, antibody-antigen, etc., wherein one member of the binding pair is labeled and is detected by binding to the other member of the binding pair. Detection of any of the above labels may be qualitative and/or quantitative.

Detection of topologically linked polynucleotides may also be via amplification of the bound polynucleotide molecule. Any amplification method known in the art may be used, so long as it yields a detectable amount of amplified product. Amplification may include, for example, by rolling circle amplification and/or polymerase chain reaction. As used herein, “amplification” refers to the process of producing multiple copies of a desired nucleic acid sequence or its complement. “Multiple copies” means at least two copies. A “copy” does not necessarily have to have perfect complementarity or identity to the template sequence. For example, copies can include nucleotide analogs such as deoxyinosine, intentional sequence alterations, for example introduced via a primer, and/or sequence errors that occur during amplification. “Rolling circle amplification” refers to an amplification process whereby circularized polynucleotide molecules of the invention that are topologically linked to a target are amplified. Circularized polynucleotide molecules of the invention that are topologically linked to a target can be isolated by affinity (hybridization) capture of the target and subsequent synthesis of a long, single-stranded copy of the circular polynucleotide by a polymerase, typically reverse transcriptase, moving around the circle multiple times in a rolling circle scheme. An example of rolling circle amplification process for polynucleotides of the invention is provided in Example 13 below. Other suitable amplification procedures are well known in the art, such as, for example, polymerase chain reaction (PCR), RT-PCR, or isothermal amplification methods. The isothermal methods include displacement amplification (Spargo et al., 1996), transcription-mediated amplification (Pastemack et al., 1997), self-sustained sequence replication (3 SR) (Mueller et al., 1997), nucleic acid sequence based amplification (NASBA) (Heim et al., 1998), an assay based on the formation of a three-way junction structure (Wharam et al., 2001), ramification amplification (Zhang et al., 2001), loop-mediated amplification (LAMP) (Endo et al., 2004; Nagamine et al., 2002).

Methods of Preparation of Allosterically-Regulatable Polynucleotide Lasso Molecules

Allosterically-regulatable polynucleotides as described above may be prepared by any method known in the art for preparation of polynucleotide molecules. For example, the polynucleotides may be prepared synthetically or expressed from an expression vector.

Polynucleotides of the invention may be prepared synthetically using methods that are well known to those of skill in the art, including, for example, direct chemical synthesis by methods such as the phosphotriester method of Narang et al. (1979) Meth. Enzymol. 68: 90-99, the phosphodiester method of Brown et al.(1979) Meth. Enzymol. 68: 109-151, the diethylphosphoramidite method of Beaucage et al. (1981) Tetra. Lett., 22: 1859-1862, or the solid support method of U.S. Pat. No. 4,458,066. Synthetic methods may be used to produce polynucleotides that contain deoxyribonucleotides, ribonucleotides, and/or modified forms or analogs thereof.

Polynucleotides of the invention may also be prepared via transcription from an expression vector. Transcription may be in vitro or may occur in vivo in an appropriate host cell. A nucleic acid encoding an allosterically-regulatable polynucleotide of the invention can be incorporated into a recombinant expression vector in a form suitable for in vitro or in vivo expression. As used herein, an “expression vector” is a nucleic acid which includes appropriate sequences to facilitate expression (e.g., replication or transcription) of an incorporated polynucleotide of interest. For in vivo expression, an expression vector can be introduced into an appropriate host cell. An expression vector may include transcriptional regulatory elements such as promoters, e.g., the T7 promoter, and/or enhancers and/or other expression control elements (e.g., polyadenylation signals). Such sequences are known to those skilled in the art (see, e.g., Goeddel (1990) Gene Expression Technology: Meth. Enzymol. 185, Academic Press, San Diego, Calif.; Berger and Kimmel, Guide to Molecular Cloning Techniques, Methods in Enzymology 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY, etc.). In some embodiments, a recombinant expression vector is a plasmid or cosmid. In other embodiments, the expression vector is a virus, or portion thereof, that allows for expression of a nucleic acid introduced into the viral nucleic acid. For example, replication defective retroviruses, adenoviruses and adeno-associated viruses can be used. Preferred in vitro expression systems include, for example, run-off transcription using bacterial, T7, SP6, and T3 RNA polymerase from appropriate templates, including single-stranded DNA templates (linear and circular), double-stranded DNA templates, and-plasmid vectors. Preferred in vivo expression systems include, for example, double-stranded DNA templates and plasmid vectors having Pol II or Pol III RNA polymerase promoters, or viral, e.g., lentiviral, vectors.

Viral expression vectors may be derived from bacteriophage, including all DNA and RNA phage (e.g., cosmids), or eukaryotic viruses, such as baculoviruses and retroviruses, adenoviruses and adeno-associated viruses, Herpes viruses, Vaccinia viruses and all single-stranded, double-stranded, and partially double-stranded DNA viruses, all positive and negative stranded RNA viruses, and replication defective retroviruses. Another example of an expression vector is a yeast artificial chromosome (YAC), which contains both a centromere and two telomeres, allowing YACs to replicate as small linear chromosomes. A number of suitable expression systems are commercially available and can be modified to produce the vectors of this invention. Illustrative expression systems include, but are not limited to baculovirus expression vectors (see, e.g., O'Reilly et al. (1992) Baculovirus Expression Vectors: A Laboratory Manual, Stockton Press) for expression in insect (e.g. SF9) cells, a wide variety of expression vectors for mammalian cells (see, e.g., pCMV-Script® Vector, pCMV-Tag1, from Stratagene), vectors for yeast (see, e.g., pYepSec1, Baldari et al. (1987) EMBO J. 6: 229-234, pMFa (Kurjan and Herskowitz, (1982) Cell 30: 933-943), pJRY88 (Schultz et al., (1987) Gene 54:113-123), pYES2 (Invitrogen Corporation, San Diego, Calif.), and the like), prokaryotic vectors (see, e.g., arabinose-regulated promoter (Invitrogen pBAD Vector), T7 Expression Systems Novagen, Promega, Stratagene), Trc/Tac Promoter Systems (Clontech, Invitrogen, Kodak, Life Technologies, MBI Fermentas, New England BioLabs, Pharmacia Biotech, Promega), PL Promoters (Invitrogen pLEX and pTrxFus Vectors), Lambda PR Promoter (Pharmacia pRIT2T Vector), Phage T5 Promoter (QIAGEN), teta Promoter (Biometra pASK75 Vector), and the like.

Allosterically-regulatable polynucleotides of the invention can be expressed in a host cell. As used herein, the term “host cell” is intended to include any cell or cell line into which a recombinant expression vector for production of an allosterically-regulatable polynucleotide, as described above, may be transfected. Host cells include progeny of a single host cell, and the progeny may not necessarily be completely identical (in morphology or in total genomic DNA complement) to the original parent cell due to natural, accidental, or deliberate mutation. A host cell includes cells transfected or transformed in vivo with an expression vector as described above.

Suitable host cells include, but are not limited to, to algal cells, bacterial cells (e.g. E. coli), yeast cells (e.g., S. cerevisiae, S. pombe, P. pastoris, K. lactis,. H. polymorpha, (see, e.g., Fleer (1992) Curr. Opin. Biotech. 3(5): 486-496), fungal cells, plant cells (e,-g. Arabidopsis), invertebrate cells (e.g. insect cells such as SF9 cells, and the like), and vertebrate cells including mammalian cells. Non-limiting examples of mammalian cell lines which can be used include CHO cells (Urlaub and Chasin (1980) Proc. Natl. Acad. Sci. USA 77: 4216-4220), 293 cells (Graham et al. (1977) J. Gen. Virol. 36: 59), or myeloma cells like (e.g., SP2 or NS0, see Galfre and Milstein (1981) Meth. Enzymol. 73(B):3-46). In one embodiment, the expression system includes a baculovirus vector expressed in an insect host cell.

An expression vector encoding a allosterically-regulatable polynucleotide of the invention can be transfected into a host cell using standard techniques. “Transfection” or “transformation” refers to the insertion of an exogenous polynucleotide into a host cell. The exogenous polynucleotide may be maintained as a non-integrated vector, for example, a plasmid, or alternatively, may be integrated into the host cell genome. The term “transfecting” or “transfection” is intended to encompass all conventional techniques for introducing nucleic acid into host cells. Examples of transfection techniques include, but are not limited to, calcium phosphate precipitation, DEAE-dextran-mediated transfection, lipofection, electroporation and microinjection. Suitable methods for transfecting host cells can be found in Sambrook et al. (1989) Molecular Cloning: A Laboratory Manual, 2nd Edition, Cold Spring Harbor Laboratory press, and other laboratory textbooks. Nucleic acid can also be transferred into cells via a delivery mechanism suitable for introduction of nucleic acid into cells in vivo, such as via a retroviral vector (see e.g., Ferry et al. (1991) Proc. Natl. Acad Sci., USA, 88: 8377-8381; and Kay et al. (1992) Human Gene Therapy 3: 641-647), an adenoviral vector (see, e.g., Rosenfeld (1992) Cell 68: 143-155; and Herz and Gerard (1993) Proc. Natl. Acad. Sci., USA, 90:2812-2816), receptor-mediated DNA uptake (see e.g., Wu, and Wu (1988) J. Biol. Chem. 263: 14621; Wilson et al. (1992) J. Biol. Chem. 267: 963-967; and U.S. Pat. No. 5,166,320), direct injection of DNA (see, e.g., Acsadi et al. (1991) Nature 332: 815-818; and Wolff et al. (1990) Science 247:1465-1468) or particle bombardment (biolistics) (see e.g., Cheng et al. (1993) Proc. Natl. Acad. Sci., USA, 90:4455-4459; and Zelenin et al. (1993) FEBS Letts. 315: 29-32).

Certain vectors integrate into host cells at a low frequency. In order to identify these integrants, in some embodiments a gene that contains a selectable marker (e.g., drug resistance) is introduced into the host cells along with the nucleic acid of interest. Examples of selectable markers include those which confer resistance to certain drugs, such as G418 and hygromycin. Selectable markers can be introduced on a separate vector from the nucleic acid of interest or on the same vector. Transfected host cells can then be identified by selecting for cells using the selectable marker. For example, if the selectable marker encodes a gene conferring neomycin resistance, host cells which have taken up nucleic acid can be identified by their growth in the presence of G418. Cells that have incorporated the selectable marker gene will survive, while the other cells die.

In one embodiment, an allosterically-regulatable polynucleotide of the invention is prepared by expression by RNA polymerase II or III in the nucleus of a host cell.

Libraries

The invention also provides libraries comprising a plurality of allosterically-regulatable polynucleotides as described above, each of which comprises a target binding sequence. Each polynucleotide may also include regulatory sequence(s) which prevent circularization of the polynucleotide around the target in the absence of binding of the target binding sequence to the target. In one embodiment, each polynucleotide comprises a target binding sequence and a catalytic domain which is capable of catalytic activity to circularize the polynucleotide around the target upon binding of the target binding sequence to the target. In various embodiments, the target binding sequence, the regulatory sequence, and/or the catalytic domain are at least partially randomized.

A variety of methods for preparation of libraries of polynucleotides are well known in the art. For example, polynucleotide libraries having randomized sequence inserts may be prepared either synthetically (either as a mixture of rationally selected sequences or partially or fully random sequences, or may be derived from directed (gene- or genome-specific) libraries. Directed libraries may be prepared by nuclease digestion, e.g., using a combination of Exonuclease III/Mung bean/BsmFI-Bbs restriction (type IIS) nucleases (Pierce and Ruffner (1998) Nucleic Acids Res. 26:5093-101; WO 99/50457), or DNase I/MmeI (Shirane et al. (2004) Nat. Genet. 36:190-196), or by consecutive digestion using a mixture of HinpI-BsaHI-AciI-HpaHI-HpyCHIV-TaqaI restriction endonucleases in combination with MmeI nuclease (Sen et al. (2004) Nat. Genet. 36:183-189). Alternative approaches for preparation of directed libraries include PCR amplification of hemi-random oligonucleotides that are selected based on either direct hybridization to immobilized DNA target (WO 00/43538; Bruckner et al. (2002) Biotechniques 33:874-882) or template-dependent ligation on DNA/RNA target templates (WO 03/100100A1).

The invention also provides a method for selection of polynucleotides that are capable of circularizing and-topologically linking to a target nucleic acid molecule, comprising contacting a target molecule with allosterically-regulatable polynucleotides from a library as described above, and amplifying the polynucleotides which become topologically linked to the target.

A novel selection approach described herein, which starts with randomized libraries of Lassos, provides simultaneous selection of both accessible target sites and optimal design of the Lasso so that circularization is dependent on prior hybridization to the target. Individual members of a Lasso library may differ from one another as follows. They may contain antisense sequences complementary to different segments of the target. They may also differ in the sequence of the circularizing moieties, for example partially randomized derivatives of naturally-occuring or naturally-existing, e.g., hairpin ribozyme, or catalytic nucleic acids derived by in vitro selection. The antisense sequences may constitute either fully random or “directed,” gene-specific libraries of antisense sequences. Circularization of Lassos is may be regulated by introduction of an regulatory element, optionally also containing partially randomized sequences.

In one embodiment, any of loops 1-3 in the HPR domain (see FIG. 2) can be used for introduction of additional or modified nucleotides (for example, randomized sequences) without appreciable perturbation of the catalytically-active structure of HPR (Feldstein & Bruening, 1993; Komatsu et al., 1994; Berzal-Herranz & Burke, 1997; Kisich et al., 1999; Fedor, 2000). In addition, catalytically non-essential residues in the other parts of hairpin ribozyme domain may also be partially (semi-random) or fully randomized (random) to increase the initial pool of the Lasso sequence libraries.

Below is a partial list of possible combinations of partial/filly randomized sequences that can be used to generate an initial pool of Lassos for selection and amplification by RCA-PCR:
<defined antisense sequence> with <semi-random internal regulatory sequence>;
<defined antisense sequence> with <random internal regulatory sequence>;
<semi-random antisense sequence> with <semi-random internal regulatory sequence>;
<semi-random antisense sequence> with <random internal regulatory sequence>;
<random antisense sequence> with <semi-random internal regulatory sequence>;
<random antisense sequence> with <random internal regulatory sequence>;
<semi-random HPR sequence> with <semi-random antisense sequence> with <semi-regulatory sequence>;
<semi-random HPR sequence> with <semi-random antisense sequence> with <random internal regulatory sequence>;
<semi-random HPR sequence> with <random antisense sequence> with <semi-random regulatory sequence>;
<semi-random HPR sequence> with <random antisense sequence> with <random regulatory sequence>.

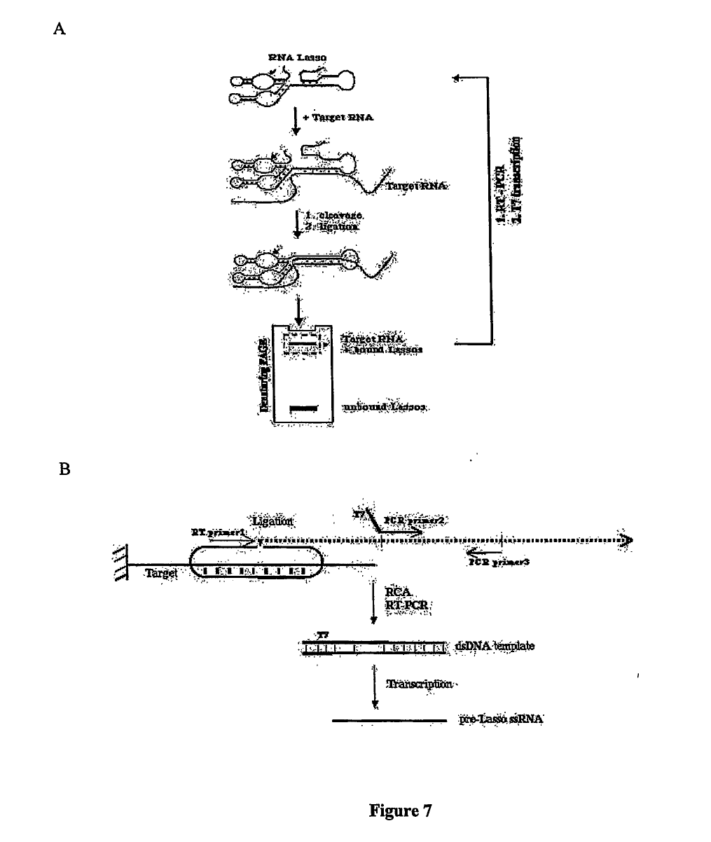

Selection with an increased number of randomized nucleotides in each of the above-mentioned regions can result in an unpredicted, but optimally effective molecular mechanism of allosteric regulation of the Lasso. In each case, the pool of Lassos is incubated with target and Lassos that can circularize in a target-dependent manner are selectively amplified by RCA-RT-PCR as described above (FIG. 7). If necessary, fine-tuning of selected sequences can be performed after the selection procedure to further optimize the efficacy of the selected sequences. This may optionally include substitution of individual residues with modified nucleotides if necessary. These modifications include but are not limited to derivatives known in the art of nucleobases, sugar residues and internucleotide bonds.

In addition, sequences in the catalytic domain (i.e., catalytically essential residues) could be further altered (rationally or using SELEX) in a way to improve the efficacy of the cleavage and ligation reactions.

Kits

The invention also provides kits that include one or more allosterically-regulatable polynucleotides or libraries as described above. Kits of the invention include separately or in combination allosterically-regulatable polynucleotides, libraries containing such polynucleotides, reagents such as buffers, expression vectors, host cells, growth medium, reagents for detection and/or amplification of topologically-linked polynucleotide-target complexes, and/or reagents for preparing libraries or arrays.

Each reagent is supplied in a solid form or liquid buffer that is suitable for inventory storage, and later for exchange or addition into a reaction or culture medium. Suitable packaging is provided. As used herein, “packaging” refers to a solid matrix or material customarily used in a system and capable of holding within fixed limits one or more polynucleotides or libraries of the invention or one or more reagent components for use with the polynucleotides, libraries, and/or methods of the invention. Such materials include glass and plastic (e.g., polyethylene, polypropylene, and polycarbonate) bottles, vials, paper, plastic, and plastic-foil laminated envelopes and the like.

In addition, the kits optionally include instructional materials providing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to Internet sites that provide such instructional materials.

Exemplification of the Invention

The invention provides design and methods for the preparation of allosterically regulated Lassos, capable of rapid, sequence-specific hybridization to nucleic acid targets and target-dependent circularization creating a strong topological link between Lasso and target. RNA Lassos containing a non-regulated hairpin ribozyme (HPR) domain that can spontaneously adopt either a linear or circular conformation have been previously described (Johnston et al., 1998, 2003). To improve the efficacy and specificity of Lasso binding to and circularization around the target, we have developed a method to make the Lasso circularize only after it binds to the target using a unique version of allosteric regulation. As proof of principle, we performed the following experiments, which are described in greater detail in the Examples below.

First, we modified the originally used hairpin ribozyme core sequence described in Feldstein & Bruening, 1993, to make it more efficient in self-processing (including both cleavage and ligation steps) according to Esteban et al. (1997). Second, we chose another antisense sequence than the one used in ATR1. This new antisense sequence corresponds to the TNFα mRNA site shown to be accessible in vivo in experiments with hammerhead ribozyme (Sioud et al., 1992, 1994, 1996; Kisich et al., 1997). Third, to make the Lasso self-processing and ligation target-dependent, we employed a scheme of allosteric regulation similar to the “TRAP”-like mechanism previously described for the cleavage reaction catalyzed by the hammerhead ribozyme (Porta & Lizardi, 1995; George et al., 1998; Burke et al., 2002. Herein, for the first time, we employed allosteric regulation for regulating the cleavage and ligation activity of the hairpin ribozyme. As with the unregulated Lasso, we used the ribozyme not for cleaving the target but rather to circularize the ribozyme around the target.

We achieved allosteric regulation by introducing complementarity between the Lasso's antisense sequence (sensor element) and another sequence of the Lasso (regulatory or inhibitory element) so as to form a regulatory complex-that prevents circularization in the absence of hybridization with target RNA. The region of internal pairing on the antisense (sensor) sequence, was chosen so that the antisense sequence has higher affinity to the complementary target sequence, which serves as the effector than to the complementary regulatory sequence. Upon binding of the antisense (sensor) sequence to the target (effector) sequence, the regulatory complex was disrupted, allowing self-processing of the Lasso ends and their ligation around the target to proceed.

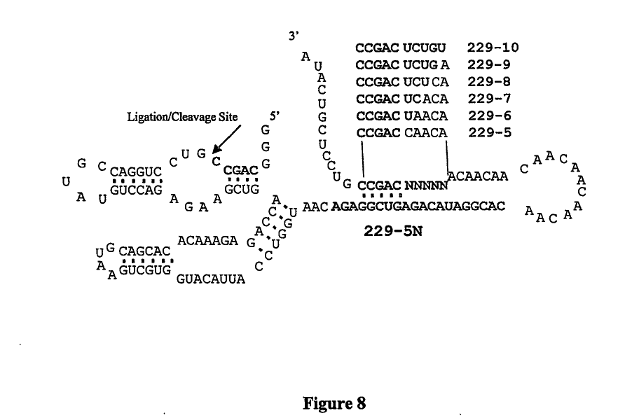

As an example of an allosterically regulated Lasso, we designed the sensor-antisense sequence to be complementary to the ‘regulatory’ element, comprising hairpin ribozyme 3′ end substrate sequence (5-nt long) extended by a few nucleotides (typically, 0 to 5 nt) into the Lasso loop sequence (FIG. 8). The presence of this regulatory structure (typically, 5 to 10 bp in length) prevents 3′ end self-processing and self-circularization in the absence of the RNA target. Since the HPR consensus sequence allows considerable sequence variability at its 3′ end substrate sequence (see FIG. 2), a large variety of regulatory structures may be rationally designed or selected from the (partially or fully) randomized sequence libraries.

A series of allosterically regulated Lassos was designed and synthesized to target the 229-249 region of murine TNFα in the long TNF2 transcript (709 nt) as well as with the short TNF-20 (20 nt long) synthetic RNA. These Lasso derivatives (ALR229-5, 229-6, 229-7, 229-8, 229-9 and 229-10) differed in the length of the regulatory elements (i.e., having 5, 6, 7, 8, 9 and 10 nucleotides complementary to the Lasso antisense domain) (FIG. 8). All of these regulatory sequences include 5 nucleotides immediately adjacent to the ribozyme cleavage/ligation site. The longer the complementarity between the regulatory element and the Lasso's antisense domain, the stronger the internal inhibition of the circularization prior the target binding. Upon binding to the TNF target, the substrate sequence could be released and the Lasso circularized as schematically shown for ALR229-8 in FIG. 9.

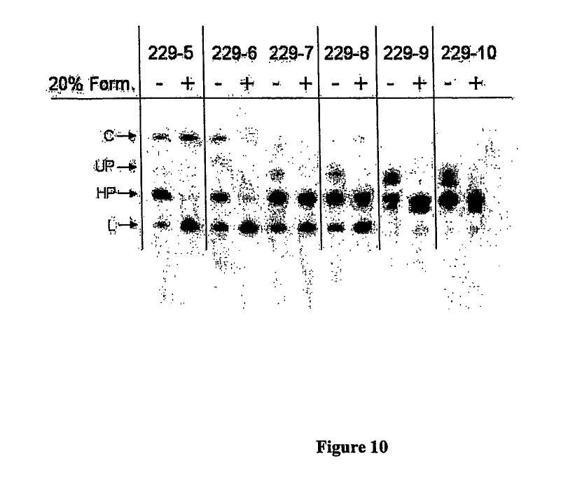

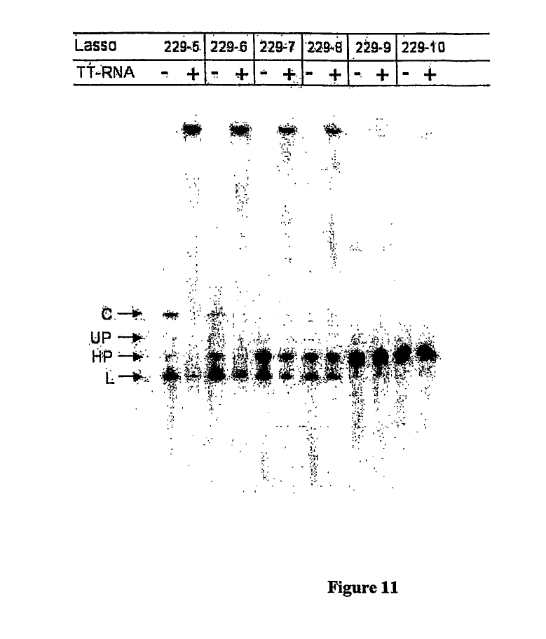

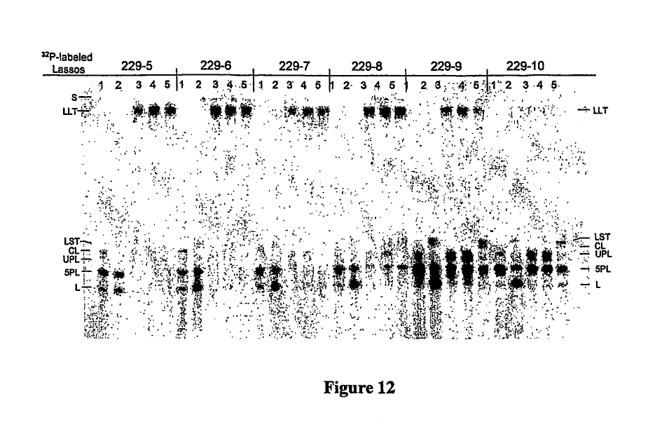

As expected, all initial Lasso transcripts underwent self-cleavage at their 5′ ends during transcription. The Lassos having the shortest regulatory sequence, ALR229-5 and ALR229-6, also processed their 3′ ends and underwent circularization, whereas 3′ end cleavage of Lassos ALR229-7 through ALR229-10 was inhibited, indicating allosteric regulation (FIG. 10). The longer regulatory elements in ALR229-9 and ALR229-10 were most effective at inhibiting processing (FIG. 10), but they also inhibited binding of these Lassos to the TNF target (FIG. 11). Overall, Lassos ALR229-6, ALR229-7, and ALR229-8 were the most effective at target binding (FIGS. 11 and 12). We found that ALR229-5 through ALR229-8 bound the long target more strongly and efficiently than the short one, and also that the Lasso-TNF2 complexes were more stable than Lasso-TNF-20 under the conditions of denaturing PAGE (FIG. 12, lanes 2-3). The superior stabilities of the [Lasso-TNF2] complexes were also confirmed by chase experiments. We found that short sense or antisense RNAs, (identical or complementary to the TNF-α target site) could not displace the long target from the [Lasso-TNF2] complexes (FIG. 12, lanes 4-5). While not wishing to be bound by theory, the stability of Lasso-TNF2 complexes may be a result of an interlocking between the two RNA secondary structures still present in TNF2 (but not in TNF-20) even in the denaturing gel conditions.

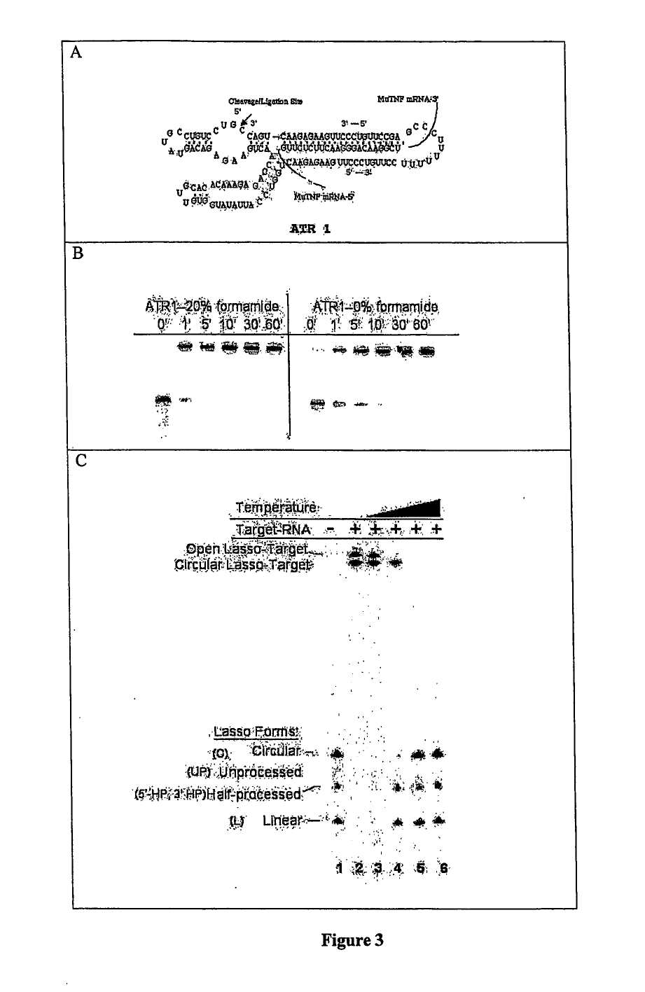

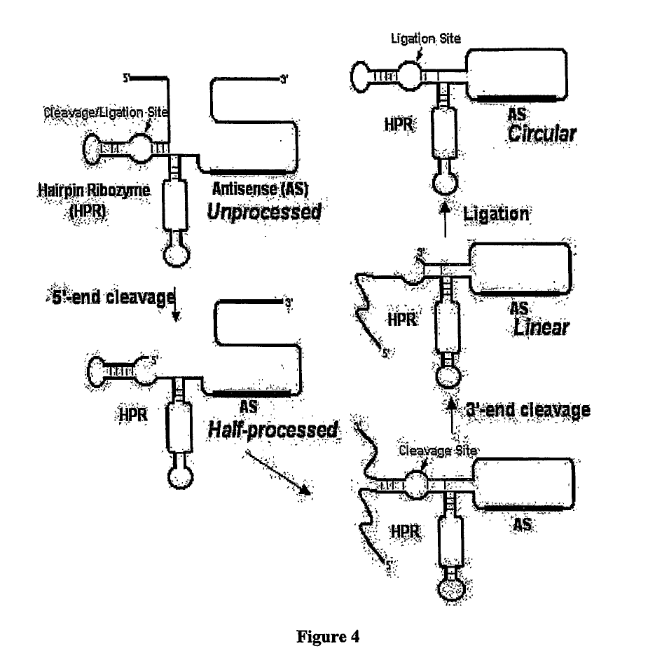

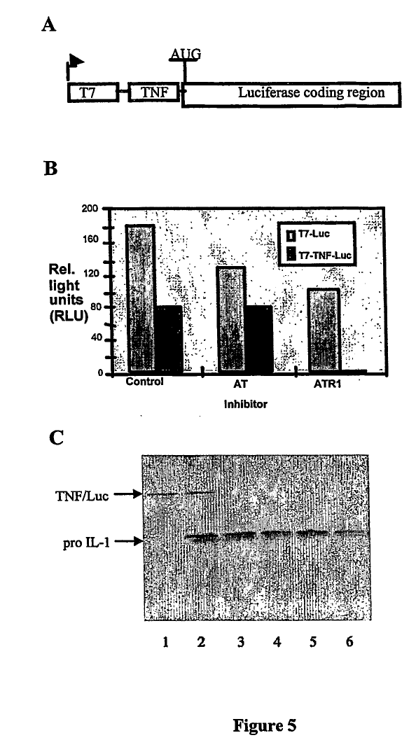

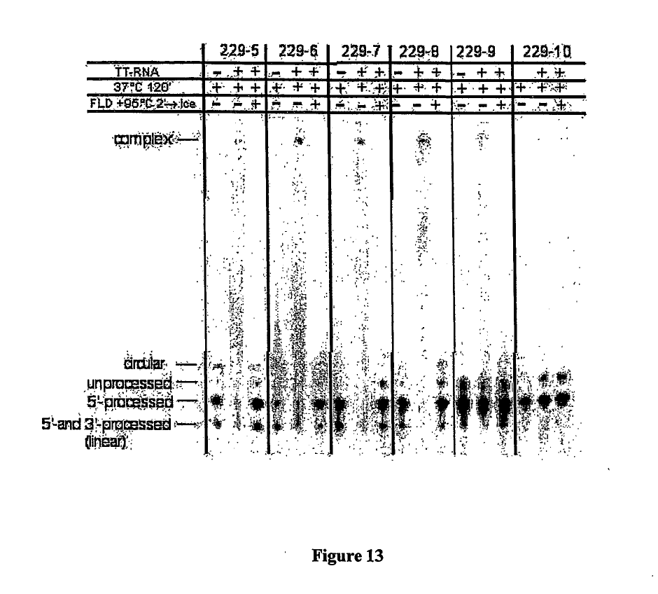

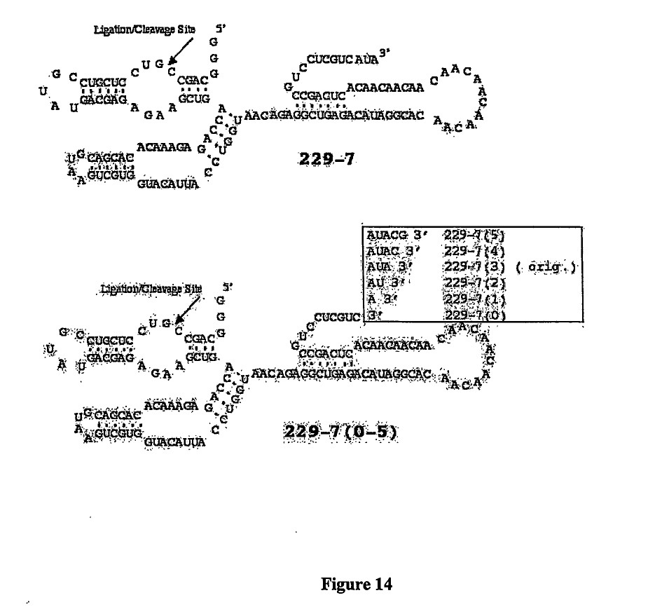

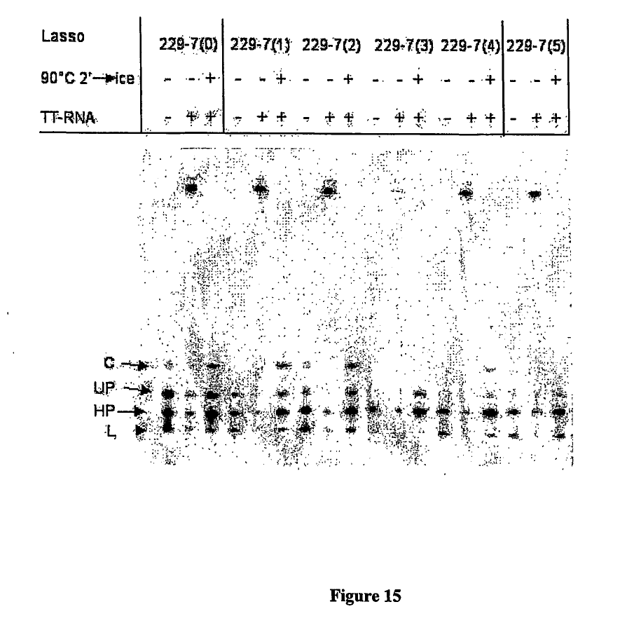

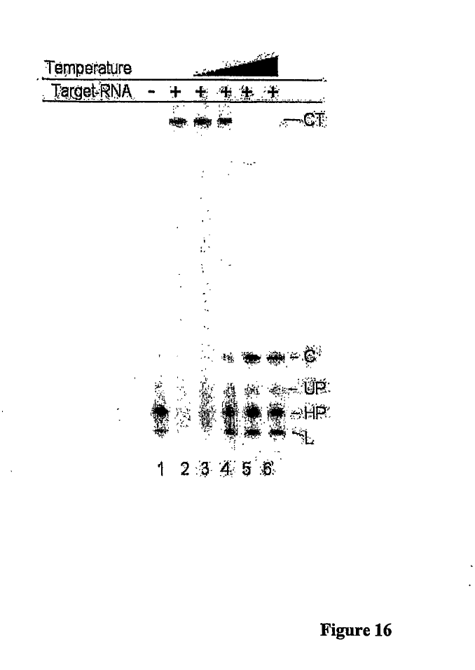

By displacing Lassos bound to TNF2 using highly denaturing conditions (60% formamide/10 mM EDTA, 95° C.), we detected the circularization of ALR229-7-8-9 Lassos induced by target binding albeit not in great yield (FIG. 13). The efficacy of target-dependent circularization was further optimized by rational tuning of Lasso sequences. Based on the experiments described above, we selected Lasso 229-7 as the best performing allosteric Lasso candidate. A series of Lassos, 229-7(0-5), with altered sequences at their 3′-ends ends, which as HPR substrate sequences, were prepared to improve the yield of circular molecules (FIG. 14). The changes in length of the Lasso 3′-ends for these constructs have modulated affinities for the HPR enzymatic domain. Lassos 229-7(0-5) were assayed for both ability to bind to target RNA and to undergo target-dependent circularization (FIG. 15). Decreasing the length of the complementarity of the 3′ end of the Lasso promoted a higher level of circularization while maintaining allosteric regulation with 229-7(0) showing the highest yield of circular species. When Lasso 229-7(0) was incubated alone, only half-processed and some fully-processed linear species were observed (FIG. 16, lane 1). After complex formation and dissociation by heat, a significant accumulation of circular Lasso species was seen (FIG. 16, lane 5). All Lasso species were gel-shifted in the presence of the target RNA, but in contrast with the ATR1 gel shifting results (FIG. 3), the complexes of the target with circular and linear Lasso species had similar gel mobility. As with ATR1, when the post-complex formation samples were heated, we observed that the linear Lasso species dissociated from target RNA at lower temperatures than circular. The reappearance of the linear Lasso species in FIG. 16, lane 4, correlates with the disappearance of the diffuse smear observed in lanes 2 and 3. The amount of Lasso observed in the CT band was the same as in the circular species band (lanes 5 and 6) after dissociation of the complex. Therefore, we infer that the strong complex band CT consists of circular Lasso RNA bound to target RNA:

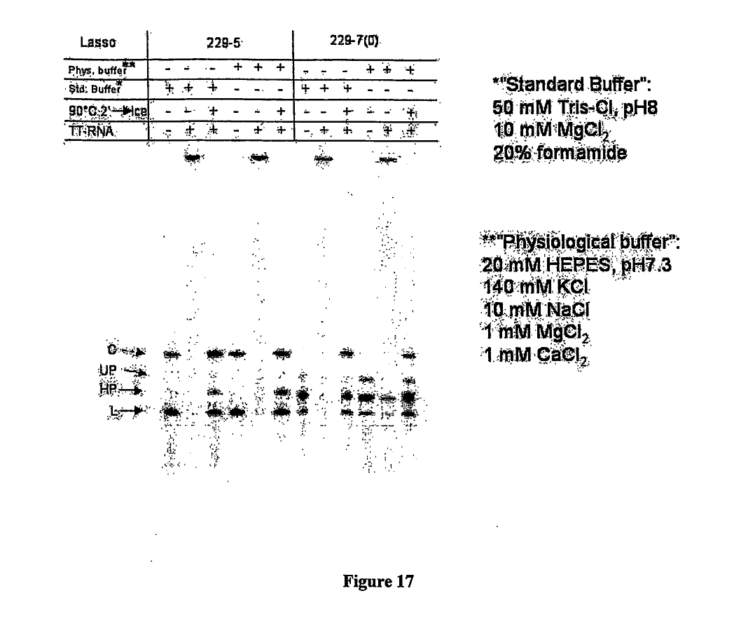

Allosterically-regulated-Lasso RNAs.(e.g. 229-7(0)) are active under a wide variety of buffer conditions. When target-binding and target-dependent circularization was tested in buffer conditions considered to be more physiologically relevant (20 mM HEPES, pH 7.3, 140 mM KCl, 10 mM NaCl, 1 mM MgCl₂, 1 mM CaCl₂) than our standard buffer conditions (50 mM Tris-Cl pH 8, 10 mM MgCl₂, 20% formamide), 229-7(0) was capable of target-dependent circularization and bound to target TNF2 (FIG. 17). Therefore, circularization of 229-7(0) after incubation with the target RNA demonstrated that the Lassos were able to circularize in conditions with low divalent cation concentration such as is present in intracellular conditions.

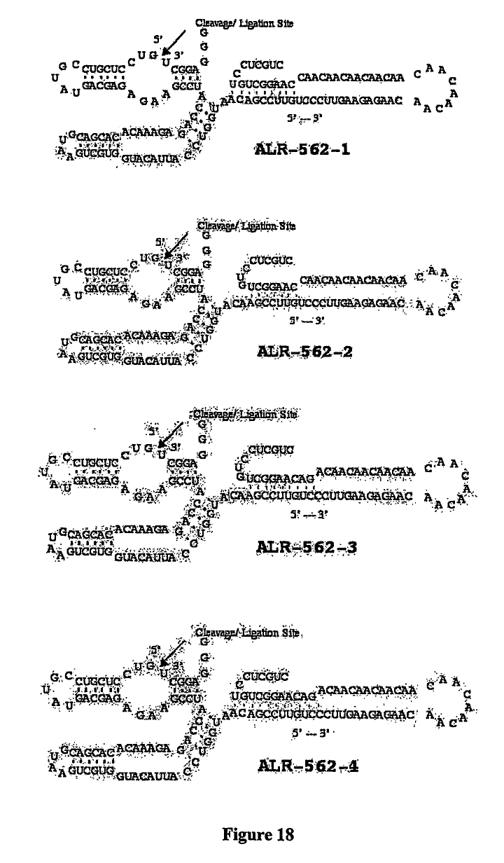

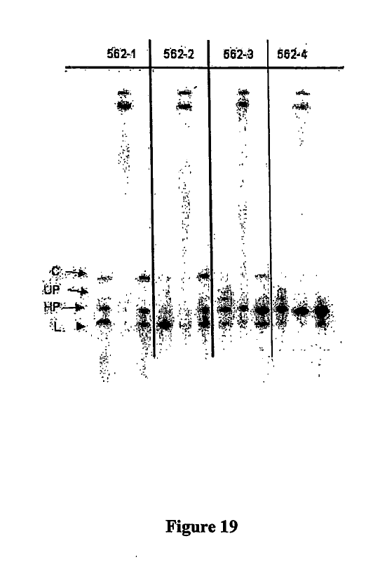

Allosterically-regulated Lassos were rationally designed to bind to other target sites. For example, we designed allosterically-regulated Lassos that bound to the target TNF 562-583. We designed several Lassos with different lengths of the regulatory sequences (ranged between 7 and 10 nt) identified as ALR562-1 through 562-4) (FIG. 18). When Lassos were assayed for TNF target binding (FIG. 19), ALR562-2, having a 7 nt regulatory sequence, showed both efficient target binding and target-dependent circularization. However, the mechanism of allosteric regulation in ALR562-2 was different than in Lasso 229-7 series, because only the Lasso circularization (but not 3′-end processing) was regulated (FIG. 19). This result demonstrates that different allosteric regulation mechanisms could be achieved while using the similar principle in design of the regulatory elements.

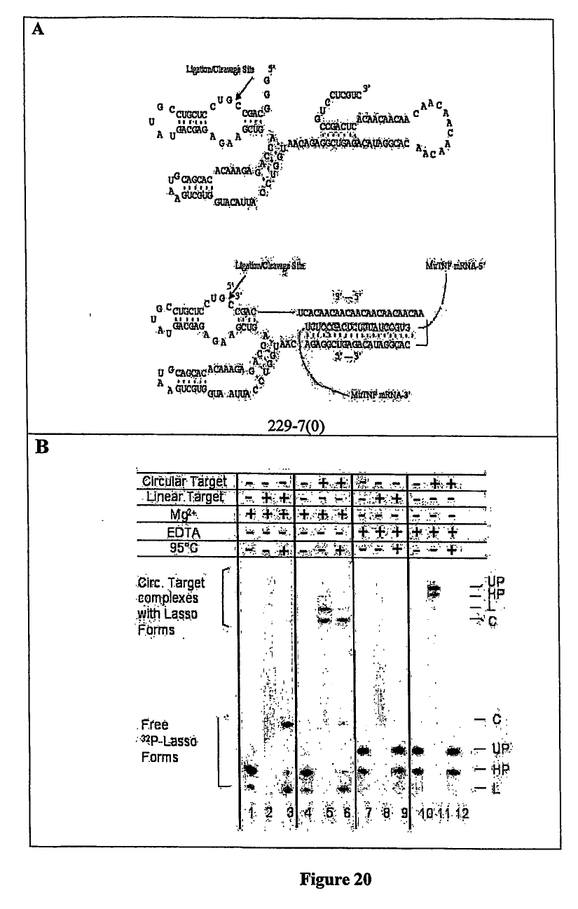

To prove that Lasso circularization upon binding to the RNA target does, in fact, result in topological link formation, we analyzed binding of the allosterically regulated Lasso 229-7(0) (FIG. 20A) with either linear or circular 120-nt model targets containing the TNF 229-248 site by denaturing PAGE. The identity of each band corresponding to different Lasso species and Lasso-target complexes was assigned analysis before and after highly denaturing treatment at 95° C. for 2 min. We found that when this Lasso was incubated with linear target RNA, it underwent efficient target-dependent circularization in the present of Mg²⁺ (FIG. 20B, lanes 1-3), whereas neither the 3′-end processing nor circularization occurred in EDTA-containing buffer (FIG. 20B, lanes 7-9). When Lasso 229-7(0) was incubated with circular target RNA without the heat-denaturation, four discrete gel-shifted bands were observed (FIG. 20B, lanes 5 and 11), whereas when this Lasso was incubated with the linear target, the corresponding Lasso-target complexes dissociated during denaturing electrophoresis and were visible only as a smear (FIG. 20B, lanes 2 and 8). Upon incubation of 229-7(0) with the circular target in buffer containing EDTA that renders the Lasso-embedded HPR catalytically inactive, two complexes with higher mobilities were observed. We found that the Lasso complexes with the short linear target were not as stable as those with longer targets (see above), possibly because the more extensive secondary and tertiary structures formed by larger target RNAs stabilize the Lasso-target complex. For the EDTA-containing reactions with circular target, the dissociated Lasso species correlate with the unprocessed and half-processed Lasso species (FIG. 20B, lanes 11-12). For the Mg²⁺-containing buffer, the upper-shifted band dissociated upon the heating, whereas the low-shifted band survived even prolonged (for up to 10 min) incubation at 95° C. (FIG. 20B, lanes 5-6). Since a circular Lasso band is not seen as a product of dissociation, the surviving band appears to represents a topologically linked complex between circular Lasso and circular target.

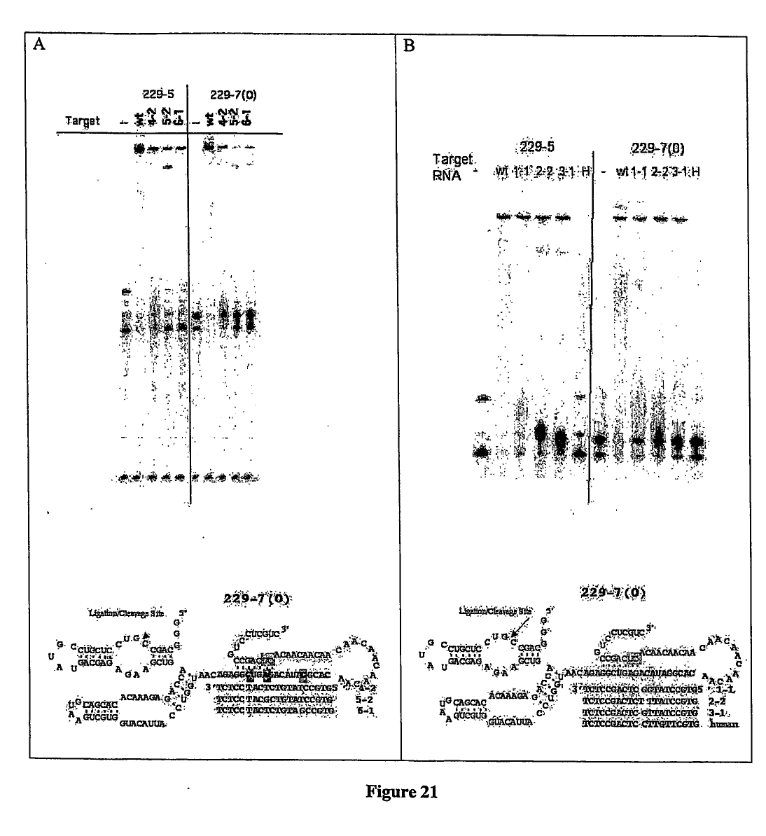

Single and multiple mutations were introduced into MuTNF targets by site-directed mutagenesis to demonstrate that allosterically-regulated Lassos confer higher sequence-specificity in comparison to non-allosterically regulated Lassos. When mismatches to target sequences occurred at nucleotide positions complemented by the regulatory element, little or no complex formation was detected for the allosterically regulated Lasso 229-7(0). However, 229-5, which is not allosterically regulated, formed complexes with the mutated target RNAs that were stable under denaturing gel electrophoresis (FIG. 21).

To both select and amplify circular molecules from a pool of RNA that contains both linear and circular RNAs, we developed a procedure involving RCA (rolling circle amplification) and RT-PCR steps (FIG. 7). This RCA-RT-PCR amplification could be used for both detection of specific targets and the selection of optimal Lasso constructs that bind these target quickly and circularize around the target efficiently. This selection procedure has been developed using a scheme of amplification of the sequence of circularized Lasso molecules by RT-PCR followed by Lasso transcription (FIG. 7).

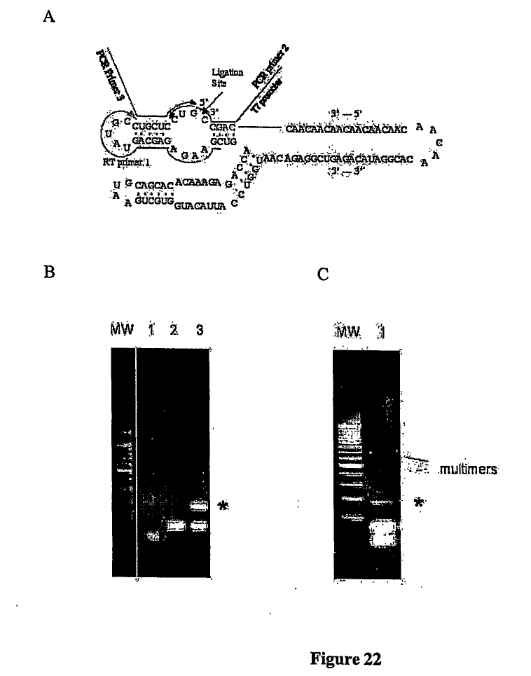

In this scheme, primer 1, which is complementary to the 5′ end of the 5′-processed Lasso, hybridizes across the active site of the HPR domain, thus inhibiting its catalytic activity and preventing further processing of the Lasso during subsequent manipulations even in the presence of Mg²⁺ ions. In the reverse transcription (RT) reaction, primer 1 selectively extends only circular Lassos, yielding single-stranded DNA multimers of the Lasso sequence (via RCA). Two additional primers (primer 2 and primer 3) are then used to amplify the RCA product by PCR and to add the T7 promoter sequence at the Lasso's 5′ end so that the products may be transcribed in vitro. As proof of principle, we were able to selectively amplify the circular form of Lasso 229-5 using this technique. In FIG. 22B, the products of RT-PCR are shown along with appropriate controls. This PCR product (marked by an asterisk in FIG. 22B) was purified by electrophoresis on an agarose gel, and the resulting template was used for in vitro transcription to confirm that an active Lasso was synthesized (data not shown). As shown in FIG. 22C, the experiment was repeated for a Lasso-target complex that was gel purified and subsequently amplified by RCA-RT-PCR. It was found that if the PCR reaction was carried out for 15 cycles, multimeric products were observed as would be predicted. As the number of cycles was increased, the monomeric form of the Lasso dominated the products of the PCR reaction. Therefore, we showed that the circularized Lassos could be selectively (in contrast to the linear, unligated Lassos) amplified by rolling circle amplification (RCA) by reverse transcription (RT) and further by PCR.

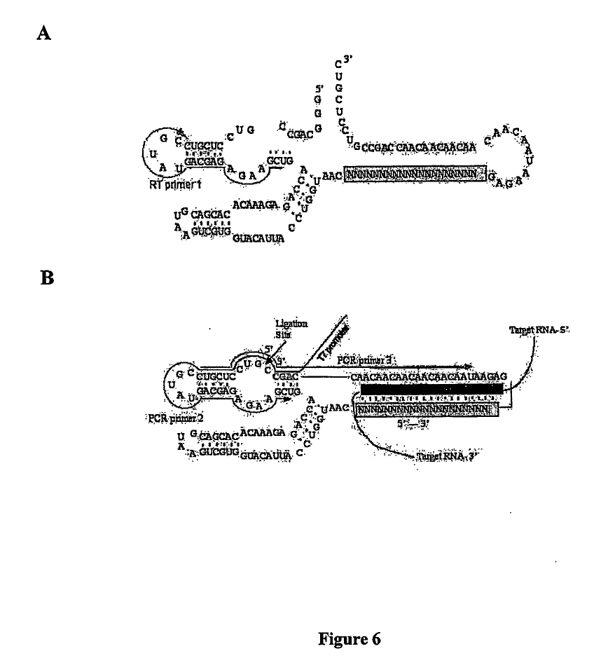

We carried out a selection using the scheme shown in FIG. 7 on a library of Lassos containing a completely randomized target binding region. We prepared a DNA template encoding the Lasso library containing the randomized antisense region and subjected it to in vitro transcription. The transcribed RNA Lasso library was then exposed to TNFα mRNA targets. The resulting strong Lasso-target complexes were isolated after separation from unbound Lasso species by denaturing gel electrophoresis. The circularized Lasso molecules bound to the target were selectively amplified by RT-PCR using specially designed primers (FIG. 6). The resulting PCR products were used as templates for transcription of RNA Lassos for another round of target binding and selection (see FIG. 7). After several rounds of selection and amplification, the DNA templates were cloned and sequenced. The selected RNA Lassos' sequences were re-synthesized and tested for their ability to tightly and specifically bind the target in vitro and to inhibit translation both in in vitro extracts and in cultured cells. After the rational optimization of the selected sequence for optimal sequence specificity (if necessary), Lasso constructs may be used as tools for target validation and gene function analysis or as antiviral, antibacterial, or gene-therapy drugs.

Although the foregoing invention has been described in some detail by way of illustration and examples for purposes of clarity and understanding, it will be apparent to those skilled in the art that certain changes and modifications may be practiced without departing from the spirit and scope of the invention. Therefore, the description should not-be construed as limiting the scope of the invention, which is delineated by the appended claims.

All publications, patents, and patent applications cited herein are hereby incorporated by reference in their, entireties for all purposes to the same extent as if each individual publication, patent, or patent application were specifically and individually indicated to be so incorporated by reference.

### EXAMPLES

The following examples are provided to illustrate but not limit the present invention.

### Example 1

### Preparation of Series of Allosterically Regulatable Lassos with Varying Allosteric Regulation Element from 5-10 Base Pairs and Assessment of Target-Dependent Circularization

Construction of DNA Template for in vitro Transcription of Lassos

A series of six Lassos containing from 5-10 internal base pairs to the antisense sequence (nt 229-248 of murine TNFα RNA) were constructed (229-5,229-6,229-7, 229-8, 229-9,229-10), as shown in FIG. 8. For each Lasso, four overlapping DNA oligonucleotides were used. Two overlapping, internal oligonucleotides were annealed and overhangs were filled in by Klenow extension. The other two oligonucleotides were primers used to amplify the, rest of the sequence by PCR.

Two partially complementary overlapping oligonucleotides were used for 229-5 through 229-10 as follows (shown in 5′-3′ direction):

(SEQ ID NO:1)

CGTCCGTATGACGAGAGAAGCTGACCAGAGAAACACACGACGTAAGTCGT
GGTACATTACCTGGTAACAGAGGC (74 nt)

(SEQ ID NO:2)

TGTTGTTGTTGTTGTTGTTGTGCCTATGTCTCAGCCTCTGTTACCAGGTA
ATGTACCACGACTTACGTC (69 nt)

The oligonucleotides were annealed at 80° C. for 5 minutes and slowly cooled to room temperature over the course of an hour. The oligonucleotides were filled in by Klenow extension to create a double-stranded template. Primers used to amplify this sequence using PCR and to add a T7 promoter sequence were as follows:

Forward PCR primer (Lassos 229-5 through 229-10):

(SEQ ID NO:3)

TAATACGACTCACTATAGGGCAGCCGTCCTCGTCCGTATGACGAGAGAAG
C (51 nt)
Reverse primers:

(SEQ ID NO:4)

TATGACGAGGACGGCTGGTTGTTGTTGTTGTTGTTGTTGTTGTGC
(229-5)

(SEQ ID NO:5)

TATGACGAGGACGGCTGATTGTTGTTGTTGTTGTTGTTGTTGTGC
(229-6)

(SEQ ID NO:6)

TATGACGAGGACGGCTGAGTGTTGTTGTTGTIGTTGTTGTTGTGC
(229-7)

(SEQ ID NO:7)

TATGACGAGGACGGCTGAGAGTTGTTGTTGTTGTTGTTGTTGTGC
(229-8)

(SEQ ID NO:8)

TATGACGAGGACGGCTGAGACTTGTTGTTGTTGTTGTTGTTGTGC
(229-9)

(SEQ ID NO:9)

TATGACGAGGACGGCTGAGACATGTTGTTGTTGTTGTTGTTGTGC
(229-10)

PCR products were purified on 1.6% agarose gel. These gel-purified fragments were used as templates for in vitro run off transcription by T7 RNA Polymerase.

In Vitro Transcription of Lassos

Lassos were in vitro transcribed using T7 RNA polymerase (Promega) for 3-5 hours at 37° C. using [³²P-α]CTP in the transcription mixture. Transcripts were desalted over a G50 micro-spin column (Amersham) and were stored at −20° C. until further use.

### Example 2

### Lasso Self-Processing and Target Binding Assays

Assays were performed using internally-radiolabeled Lassos, incubated either alone or with an excess of TNF2 target RNA (cold) at 37° C. for 120 minutes in one of three buffers: (i) 50 mM Tris-HCl, pH 7.5, 10 mM MgCl₂; (ii) 50 mM Tris-HCl, pH 7.5, 10 mM MgCl₂, 20% formamide volume/volume; (iii) 20 mM HEPES, pH 7.3, 140 mM KCl, 10 mM NaCl, 1 mM MgCl₂, 1 mM CaCl₂. Reactions were quenched with an equal volume of loading buffer containing 90% formamide, 10 MM EDTA, 0.01% bromophenol blue, 0.01% xylene cyanol. Samples were analyzed on 6% PAGE/8M urea/0.5× TBE gels and were electrophoresed at 11 Watts for approximately two hours. Gels were dried and either directly scanned by phosphorimager or exposed to X-ray film.

### Example 3

### Self-Processing Activity of Allosterically Regulated Lassos and Effect of 20% Formamide in the Processing Buffer

Lassos 229-5 and 229-6 (five and six base pair regulatory sequences, respectively) were able to fully self-process both 5′ and 3′ ends as evidenced by linear and circular gel bands (FIG. 10). Lassos 229-7 through 229-10 did not circularize when incubated without target RNA, and so were allosterically regulated. The presence of 20% formamide in the assay buffer improved Lasso self-processing (FIGS. 10 and 11).

### Example 4

### The Effect of Increased Internal Base Pairing on Target Binding Ability or Determination of Optimally Allosterically Regulated Lassos

Lassos as described in Example 1 were tested in target binding assays as described in Example 2 in buffer containing 20% formamide. As the length of the regulatory sequence was increased, the efficiency of target binding decreased, while the extent of target-independent processing was reduced (FIG. 11). There was a tradeoff between the “level” of allosteric regulation and efficiency of target binding for each Lasso. For Lassos targeting the, 229 region of murine TNFα, the optimal regulatory length was determined to be seven or eight base pairs because both efficient target binding and the prevention of circularization of Lassos prior to target binding were observed with base paired regulatory sequences of these lengths.

### Example 5

### Comparison Between Short and Long Target RNAs

Lassos 229-5-6-7-8-9-10 were transcribed from DNA templates and tested in binding experiments with the long TNF2 target as well as with the short 20 nt synthetic TNF-20 RNA (Dharmacon) comprising just 20-nt of target TNF sequence (FIG. 12). Internally ³²P-labeled Lassos were incubated in 10 mM MgCl₂/50 mM Tris-Cl (pH 8) for a total of 120 minutes at 37° C., either alone (lanes 1) or with non-radioactive 0.4 μM TNF-20 (lanes 2) or non-radioactive 0.4 μM TNF2 (lanes 3-5). Lanes 4 are the same as lanes 3 but chased with a 14-fold excess of 20-nt competitor antisense RNA, anti-TNF-20 over TNF2. Lane 5 is the same as lane 3 but chased with 7-fold excess of competitor sense TNF-20 (20-nt) over TNF2. Samples were analyzed by 6% denaturing PAGE. Anti-TNF-20 is identical to the antisense sequence incorporated into the Lassos. TNF-20 corresponds to the sequence of TNF-α mRNA targeted by these Lassos.

Lasso transcripts (pre-Lassos) underwent self-cleavage at their 5′-ends during transcription, whereas the cleavage of their 3′-ends was inhibited (see FIG. 12). ALR229-9 and ALR229-10 self-cleave their 5′-ends less efficiently than the other Lassos (during both transcription and incubation in the presence of TNF2, but not if incubated alone or in the presence of TNF-20). We found that inhibition increased with increasing length of the regualatory elements (FIG. 12, lanes 1). We demonstrated that Lassos ALR229-6-7-8-9-10 indeed underwent allosteric regulation upon binding to the target sequence. The target binding allowed the ribozyme to complete self-processing, yielding fully processed linear Lassos (FIG. 12, lanes 2).

We showed that the ALR229-5-6-7-8 Lassos bound the long target more strongly and more efficiently than the short target, and also that the Lasso-TNF2 complexes were more stable than Lasso-TNF-20 under the conditions of denaturing PAGE (FIG. 12, lanes 2-3). Overall, ALR229-6-7-8 Lassos were the most effective at target binding. The superior stabilities of the Lasso-TNF2 complexes were also confirmed by chase experiments. We found that short sense or antisense RNAs, (identical or complementary to the TNF-α target site) could not displace the long target from the Lasso-TNF2 complexes (FIG. 12, lanes 4-5). Although not wishing to be bound by theory, the increased stability of Lasso-TNF2 complexes may be a result of an interlocking between the two RNA secondary structures still present in TNF2 (but not in TNF-20) even under denaturing gel conditions.

### Example 6

### Assay for Target-Dependent Circularization

Lasso-target complexes were formed and quenched as described in Example 2. To test for target-dependent circularization, half of the Lasso-target complex reaction was heated in loading buffer for 2 minutes at 90° C. and then placed immediately on ice to prevent complex re-hybridization prior to loading on a denaturing gel. Lasso incubated without target RNA, undissociated complex and dissociated complex were loaded in adjacent lanes on 6% denaturing gel. Lasso species (dissociated) were compared to Lasso species present before incubation with target RNA to assess the extent of target-dependent circularization.

Complexes were formed with Lassos 229-5 through 229-10 without formamide present in the incubation buffer. Lassos 229-7, 229-8, and 229-9 show an accumulation of circular Lasso species that was not present when the Lasso was incubated without target. 229-5 and 229-6 contained some circular species before incubation with target and did not show allosteric regulation. After heat treatment, circular form remained, but not more than was originally present. 229-10 did not form complex with target RNA under these conditions. (FIG. 13).

### Example 7

### Optimization of the 3′ Ends of Lassos

A series of Lassos were designed with altered 3′ ends to improve the ability of allosterically regulated Lasso 229-7 to circularize (229-7(0-5)) (FIG. 14). 229-7(0-5) Lassos were assayed for ability to bind to target RNA and for circularization upon complex formation (FIG. 15). All of the Lassos were able to bind to target TNF2 efficiently, but the amount of circularization upon target binding decreased as the length of the complementarity of the 3′-end to the helix/loop 1 region of the hairpin ribozyme domain of the Lasso increased. Lassos 229-7(0,1, and 2) showed an improvement in the amount of Lasso that had circularized after being incubated with TNF2 than the original 229-7(3). A comparison between Lassos 229-7(0) and 229-7(3) in buffer containing or lacking 20% formamide was performed (data not shown). Circular RNA was produced even under the more denaturing conditions. Decreasing the length of the complementarity of the 3′ end of the Lasso promoted a higher level of circularization while maintaining allosteric regulation.

### Example 8

### Target-Dependent Circularization of an RNA Lasso and Temperature Dissociation

The rationally designed and tested RNA Lasso 229-7(0) has partially self-complementary antisense domains, and was demonstrated to have target-dependent circularization ability with respect to a pre-selected accessible site on TNFα RNA. When Lasso 229-7(0) was incubated alone, only half-processed and some fully-processed linear species were observed (FIG. 16, lane 1). After complex formation and dissociation by heat, a significant accumulation of circular Lasso species was seen (FIG. 16, lane 5). As with ATR1, when the post-complex formation samples were heated, we observed that the linear Lasso species dissociated from target RNA at lower temperatures than circular (compare lanes 4 and 5). The reappearance of the linear Lasso species in FIG. 16, lane 4, correlates with the disappearance of the diffuse smear observed in lanes 2 and 3. The amount of ³²P-labeled Lasso observed in the CT complex band was the same as in the circular species band (lanes 5 and 6) after dissociation of the complex. Therefore, we infer that the strong complex band CT consists of circular Lasso RNA bound to target RNA.

### Example 9

### Allosteric Regulation and Lasso Processing Under Different Buffer Conditions

Lasso 229-7(0) was shown to be capable of target-dependent circularization under buffer conditions considered to be more physiologically relevant (20 mM HEPES, pH 7.3, 140 mM KCl, 10 mM NaCl, 1 mM MgCl₂, 1 mM CaCl₂) than standard assay buffer conditions (50 mM Tris-Cl pH 8, 10 mM MgCl₂, 20% formamide). Under these conditions, Lassos 229-5b and 229-7(0) bound efficiently to target TNF2 and showed similar amounts of circularization after incubation at 37° C. for 120 minutes and subsequent complex displacement by 95° C. treatment as was observed for the standard buffer (FIG. 17). (Lasso 229-5b is a variant of Lasso 229-5. The only difference in the sequence between 229-5 and 229-5b is that there is an additional three nucleotides (5′-AAC-3′) inserted directly 5′ to the antisense sequence. 229-5b has a 5 base pair regulatory element as 229-5 does, whereas 229-7(0) has a seven base pair regulatory element.) Circularization of 229-7(0) after incubation with the target RNA demonstrated that the Lassos were able to self-ligate in conditions with low divalent cation concentration. It should be noted that for the allosterically regulated Lasso 229-7(0), circularization was completely dependent on the presence of target whereas for the non-allosterically regulated Lasso 229-5b, circularization occurred in the absence of target RNA.

### Example 10

### Lassos Directed Towards a TNF Target Site

A series of allosterically regulated Lassos was designed to target the TNF 562-583 sequence in the coding region. The regulatory sequences in these Lassos ranged between seven and ten base pairs (FIG. 18). The Lassos were assayed for target binding and target dependent circularization (FIG. 19). All of these new Lassos circularized when incubated with target RNA, and all bound to target RNA strongly. 562-2, which has a seven base pair masking sequence circularized very efficiently only when incubated with target RNA. However, the mechanism of allosteric regulation was different than other allosterically regulated Lassos because it almost fully processed to linear (5′ and 3′ processed) upon incubation in standard buffer without target. The equilibrium of the Lasso processing was such that the 3′ end was able to process efficiently but ligation did not occur until the Lasso was incubated with the target RNA. Therefore, different allosteric regulation mechanisms are possible although the design of the regulatory element is the same.

### Example 11

### Topological Linkage of Allosterically Regulated 229-7(0) to Circular Target RNA

A 120 nt circular RNA target containing the TNF 229-248 nt site was prepared using the strategy described by Beaudry and Perreault (1995). ³²P-labeled Lasso 229-7(0) was incubated with linear and circular targets, respectively, under conditions where Lassos can (i.e., in the presence of Mg²⁺) (FIG. 20B, lanes 1-6) and cannot (i.e., in the absence of free Mg²⁺) (FIG. 20B, lanes 7-12) self-process. When the Lasso was incubated with circular target RNA in the presence of 10 mM Mg²⁺, three discrete gel-shifted bands were observed (FIG. 20B), whereas when 229-7(0) was incubated with the linear target in both buffers, complexes dissociated during electrophoresis conditions and were visible only as a smear. Upon incubation of 229-7(0) with the circular target in buffer containing EDTA that renders the Lasso catalytically inactive, two higher mobility complexes were observed.

The identity of each complex was assigned by analyzing the products of dissociation after incubating the complexes at 95° C. for 2 min followed by quenching on ice prior to loading on the gel (see FIG. 20B). When reactions containing Lasso and linear target were heated, the Lasso was able to circularize in the presence of Mg²⁺ ions but not in the EDTA-containing buffer. The Lasso complexes with shorter linear targets were not as stable as those with longer targets, possibly because the more extensive secondary and tertiary structures formed by larger target RNAs prevent dissociation of the Lasso-target complexes. For the EDTA-containing reactions with circular target, the dissociated Lasso species correlate with the unprocessed and half-processed Lasso species. For the Mg²⁺-containing buffer, the two upper gel shift bands were mostly dissociated upon incubation at high temperature and correlate with the reappearance of fully processed and half-processed linear forms of the Lassos, respectively. One of these gel shifted bands, the lowest mobility band, survived incubation even at 95° C. for up to 10 min. Since a circular Lasso band was not seen as a product of dissociation, we concluded that the surviving band represented a topologically linked complex between an allosterically regulated circular Lasso and circular target.

### Example 12

### Increased Specificity of Allosterically Regulated Lassos for Mismatched Target RNAs

A series of mutated TNF2 targets were synthesized (Stratagene Quick Change mutagenesis kit) with mismatches to the 229-7(0) antisense sequence as shown in FIG. 21. When mismatches overlap with sequence elements in the allosteric or “sensor” element, binding to mismatched targets was greatly reduced or abolished (FIG. 21A). Binding assays were carried out with non-allosterically-regulated Lasso 229-5 and with allosterically regulated 229-7(0). Lasso 229-7(0) did not bind to targets containing two mismatches in the sensor element whereas 229-5 bound much more efficiently. When the mismatches were outside of the sensor element, both Lassos were able to bind the mismatched targets (FIG. 21B).

### Example 13

### Amplification of Circular Lasso by RCA-RT-PCR

To select and amplify only circular molecules from a pool of Lasso RNA that contains both linear and circular species, we developed a scheme that uses RCA (rolling circle amplification) in an RT-PCR reaction (shown schematically in FIG. 7).

In this scheme, a primer used to reverse transcribe only circular molecules (RT primer 1: 5′-GCTTCTCTCGTCATACG-3′ (SEQ ID NO: 10)) was annealed to the unique, complementary sequence near the 5′ end of the Lasso transcript by incubating for 1 min. at 85° C. followed by cooling at room temperature for 5 min. The primer hybridized across the active site of the HPR domain, and prevented further self-processing of the Lasso during subsequent manipulations. In the reverse transcription (RT) reaction, RT primer 1 extended only circular Lasso species selectively, yielding (via RCA) single-stranded DNA multimers of the Lasso sequence. Linear (or unligated) Lassos yielded only a short abortive product, which would not be amplified by PCR in the next step. Several commercially available reverse transcriptases were tested to optimize the procedure. Of those tested, Superscript II (Invitrogen) gave consistent, reliable rolling circle amplification. The RT reaction was carried out for 1 hour with the SuperScript II enzyme according to the manufacturer's protocol. Two additional primers (PCR primer 2: 5′-TAATACGACTCACTATAGGGCAGCCGTC-3′ (SEQ ID NO: 11) and PCR primer 3: 5′-GGTGACACTATGATGCATATGACGAGGAC-3′ (SEQ ID NO: 12)) were then used to amplify the RCA product by PCR and to add the T7 promoter sequence at the 5′-end of the Lasso so that the product could be transcribed by T7 RNA polymerase.

This technique was initially tested on the free Lasso in the absence of its target. We were able to selectively amplify the circular form of a previously characterized Lasso 229-5 (FIG. 22A), with defined antisense sequence complementary to nucleotides 229-248 of mTNFα, as proof of principle. In FIG. 22B, the products of RT-PCR are shown along with appropriate controls. This PCR product (marked by an asterisk in FIG. 22) was purified by electrophoresis on an agarose gel, and the resulting template was used for in vitro transcription to confirm that an active Lasso was synthesized (data not shown). In FIG. 22C, the experiment was repeated for a Lasso-target complex that was gel purified and subsequently amplified by RCA-RT-PCR. It was found that if the PCR reaction was carried out for 15 cycles, multimeric products were observed as would be predicted. As the number of cycles is increased, the monomeric form of the Lasso dominates the products of the PCR reaction.

### Example 14

### In Vitro Selection with a Pool of RNA Lassos Containing a Fully Randomized “Antisense” Region

Preparation of Lasso DNA Cassette Containing a 20N Randomized Target Region

Lasso DNA cassettes encoding a fully randomized 20N target sequence and T7 RNA polymerase promoter were prepared by PCR cloning using the overlapping oligonucleotide scheme described in Example 1 with the exception that the sequences corresponding to 229 antisense were replaced by 20 N randomized bases. Lasso library selection

The 20N Lasso library was transcribed in vitro with T7 RNA polymerase (Ambion) to generate an initial pool of Lassos for in vitro selection (FIG. 6A). We confirmed that the transcribed library contained active Lasso species that could self-process and circularize (data not shown). Six rounds of selection were performed with primers for RCA-RT-PCR depicted in FIGS. 6A and 6B. For the initial round of selection, 1000 pmol of the Lasso library was incubated with an excess of target RNA at 37° C. for 60 minutes in 50 mM Tris-HCl, pH 7.5, 10 mM MgCl₂, 20% formamide. These conditions ensured that the library complexity was retained through the initial round of selection. Reactions mixtures were electrophoresed on a 6% denaturing gel to separate free Lasso and free target RNA from Lasso-target complex (see schematic in FIG. 7A). RNA was visualized in the gel by ethidium bromide staining. Excised and eluted complexes from the gel slices were amplified by RCA-RT-PCR as described in Example 13. The RT-PCR product was gel purified on a 1.5% agarose gel and extracted using QIAquick Gel Extraction Kit (Qiagen). The resulting DNA was used as the transcription template to generate an enriched Lasso library for the next round of selection. The entire selection process was repeated five times with decreases in incubation time. For each round of selection, an increased amount of complex was formed from the selected pool of Lassos, indicating that the procedure enriched for sequences that interacted with target RNA faster and more efficiently.

### Example 15

### Preparation of a Small Lasso Library Comprising a Rationally Designed Hairpin Ribozyme Domain, a Randomized Regulatory Element, and a Defined Antisense Sequence

A mini-library was synthesized of ALR229-5N Lassos (FIG. 8), which contain a rationally designed hairpin ribozyme domain, a defined antisense sequence, and a hemi-random regulatory element. DNA templates for the mini-library transcription were prepared using four DNA primers. First, two overhanging primers that encode the internal Lasso region were annealed and extended by DNA Polymerase I (Klenow fragment). Two additional primers that include the flanking Lasso sequences were used to extend and amplify the resulting DNA template by PCR. The prepared Lasso DNA library, containing a 5 bp randomized region, was then transcribed to prepare a Lasso RNA library. Lasso RNAs were desalted by gel filtration (on a G-50 micro-spin column) and incubated with TNF2 target. The Lasso-target complexes were isolated, and the circularized Lasso RNAs were passed through several rounds of selection as described above.

After the last round of selection, 20 resulting DNA fragments are cloned and sequenced. The obtained sequences are compared to the related sequences of ALR229-5-6-7-8-9-10 are statistically analyzed.

### Example 16

### Selection of the Optimized Lassos from the Partially Randomized Libraries

We developed a method for detecting and amplifying only Lassos in their covalently ligated circular form. To detect the circularization of Lasso RNAs, we designed primers for RT-PCR (similar to those shown in FIG. 6) that amplify only the circular Lassos.

Primer 1 was designed to be complementary to the 5′-end of the 5′-processed Lasso. In the reverse transcription (RT) reaction, Primer I (5′-GCTTCTCTCGTCATACG-3′ (SEQ ID NO: 10)) selectively extended only circular Lassos, yielding single-stranded DNA multimers of the Lasso sequence (rolling circle amplification, RCA). Two additional primers (Primer 2 (5′-TAATACGACTCACTATAGGGCAGCCGTC-3′ (SEQ ID NO: 11)) and Primer 3 (5′-GGTGACACTATGATGCATATGACGAGGAC-3′ (SEQ ID NO:12)) were used to amplify the RCA product by PCR and to restore the T7 promoter sequence at the Lassos 3′-end so that the products could be transcribed in vitro. Since this PCR reaction sometimes yields multiple products, the DNA fragment corresponding to the monomer Lasso sequence was gel-purified. The resulting DNA template was used for transcription of selected Lasso RNAs.

The internally ³²P-labeled Lasso RNAs were incubated at 37° C. with TNF2 target (709-nt fragment of TNF mRNA) in buffer containing 50 mM Tris-Cl (pH 7.5)/10 mM MgCl₂/20% formamide (standard binding conditions). The resulting complex was isolated by denaturing PAGE. The band corresponding to the Lasso-target complex was localized on the gel by autoradiography, then excised and eluted. The eluted Lasso complexed with the target was then amplified by RT-PCR as described above.

### Example 17

### Design and Preparation of a Lasso DNA Cassette to Encode a Lasso RNA Library

The sequence of an RNA Lasso scaffold is designed to contain a partially randomized hairpin ribozyme domain, a randomized regulatory element to select for target-dependent circularization, and directed antisense sequences. Using the 229-SN Lasso mini-library as a scaffold, an RNA Lasso library comprising partially randomized ribozyme and regulatory sequences, and a directed antisense library is designed. Restriction sites (XhoI and BamHI) flank either side of a 20-nucleotide antisense cassette sequence, which is supplied by the directed library. The Lasso contains a 10 nucleotide randomized region downstream of the BamnHI site and loop, which constitutes the variable allosteric regulatory element that is optimized through iterative rounds of selection and amplification. The 5′ end of the ribozyme core is also partially randomized to allow for proper processing of the 3′ end of the Lasso molecules induced by the binding to the target. Throughout the Lasso, nucleotides essential to hairpin ribozyme activity (see FIG. 2) are preserved.

Based on the structure of the RNA Lasso library, a DNA library cassette encoding ribozyme, regulatory sequences, and restriction sites allowing the insertion of the directed antisense libraries in desirable orientation is designed and synthesized. This DNA library cassette is prepared in two halves to prevent PCR amplification of cassettes without the directed library insert. The first half contains the T7 promoter sequence, the hairpin ribozyme domain, and the XhoI restriction site. The second half includes the BamHI site, the regulatory element and the 3′-end of the Lasso. Each template segment contains an arbitrary sequence adjacent to the restriction sites to enable efficient digestion.

Directed DNA libraries encoding up to 20-nt-long sequences complementary to TNFα, flanked by restriction sites to allow insertion into the DNA library cassette, using the method described in WO 03/100100A and VLassov et al. (2004) Oligonucleotides (in press). Two hemi-random probes consisting of a defined sequence (PCR primer and restriction site sequences) and I 0-nt randomized regions are prepared. The hemi-random probes are hybridized to TNFα cDNA and adjacent probes are ligated with DNA ligase. The ligation products are amplified by PCR, followed by digestion with the appropriate restriction enzymes.

A target cDNA, comprising the sequence of the target mRNA, is prepared using plasmid PGEM-4/TNF, encoding MuTNFα, by asymmetric PCR with an unmodified primer. Alternatively, regular exponential PCR with a biotinylated primer can be used with subsequent ccDNA strands separation on streptavidin magnetic beads Dynabeads M-280 Streptavidin (Dynal, 20001). In-he first step, the dsDNA is immobilized on the beads due to biotin-streptavidin binding. The mixture is then be heated to separate the DNA chains: non-biotinylated strands appear in the flow-through, while the biotinylated strands remain attached to the beads.

Two hemi-random DNA probes are designed and synthesized, comprising sequences of the defined PCR primer (20 nt) and restriction sites, Xho I & Bam HI (6 nt), with a randomized region (10 nt). Also, masking oligonucleotides that are complementary to the constant regions of the probes are prepared. The hemi-random probes (with constant regions protected with masking oligonucleotides) are hybridized to TNF-α cDNA and the adjacent probes are ligated by T4 DNA ligase at 25-40° C. as described by Kazakov et al (2002). The ligated probes are amplified by PCR using specific primers.

The Lasso DNA library cassettes are digested with appropriate restriction enzymes, and ligated with the digested directed antisense library. The ligated products are amplified by PCR and transcribed to prepare an RNA Lasso library.

To combine the directed library with the DNA library cassette halves, two halves of the DNA library cassette and the directed library species are digested with the appropriate restriction enzymes to generate cohesive ends. The digested products are gel-purified and ligated by DNA Ligase. The ligated product is PCR-amplified using primers that are specific to the fill-length ligated product. The amplified DNA molecules are gel-purified and used as templates for transcription of the Lasso RNA library as schematically presented in FIG. 7b.

### Example 18

### Selection of the RNA Lassos Best able to Quickly Bind and Form Topological Links with Specific Targets

An RNA Lasso library as described above is incubated with target, followed by isolation of complexes on an affinity column, selective amplification of circularized Lassos from Lasso-target complexes, and transcription of RNA from the PCR products. After several additional rounds of selection, surviving members of the library are cloned, sequenced, re-synthesized and tested in the binding assays. The RNA Lasso libraries are incubated with the target TNFα mRNA. The resulting complex is isolated using biotinylated ccDNA complementary to TNFα mRNA immobilized on streptavidin-coated magnetic beads as described previously (Deyev et al., 1984; Stiege et al., 1988; Dynal, 2000). To prevent enrichment of unrelated RNAs (false-positive) that can non-specifically bind beads and undergo self-circularization in a target-independent manner, a counter-selection with is performed with blank magnetic beads or non-specific RNA target (e.g., biotinylated IL-1 ccDNA). After a brief incubation, the beads are washed intensively to remove non-bound and non-specifically bound molecules. Then, Lassos that are specifically bound to the target RNA are eluted.

The eluted Lassos complexed with the TNF target are amplified by RT-PCR as described above. If amplification proves difficult with intact Lasso-target complex, the Lasso-target complex may be dissociated prior to primer extension under highly denaturing conditions that retain the integrity of circularized Lasso. After the first round of selection, the Lasso DNA library is transcribed into the Lasso RNA library. Lasso RNAs are desalted by gel filtration (for example, on a G-50 micro-spin column) and incubated again with the target TNF-α mRNA. The Lasso-target complexes are isolated as described above, and the circularized Lasso RNAs are passed through several additional rounds of selection.

After the last round of selection, around 50-100 resulting DNA fragments are cloned and sequenced. The sequences obtained are assigned to the TNF target sequences and statistically analyzed.

### BIBLIOGRAPHY

Al-Anouti, F., and Ananvoranich, S. (2002) Antisense Nucleic Acid Drug Dev. 12, 275-81.
Allawi, H. T., et al. (2001) RNA 7, 314-327.
Amarzguioui, M., et al. (2003) Nucleic Acids Res. 31, 589-595.
Antson, D. O., et al. (2000) Nucleic Acids Res. 28, e58.
Baner, J., et al. (2001) Curr. Opin. Biotechnol. 12, 11-15.
Beaudry, D., and Perreault, J.-P. (1995) Nucleic Acids Res. 23, 3064-3065.
Beckaniam, J. S., Daniel, V. (1974) J. Mol. Biol. 89, 355-62.
Bell, S., et al. (2003) Cell. Signaling 15, 1-7.
Berzal-Herranz, and A., Burke, J. M. (1997) Methods Mol. Biol. 74, 349-355.
Bohula, E. A, et al. (2003) J. Biol. Chem. 278, 15991-15997.
Boden, D., et al. (2003) J. Virol. 77, 11531-11535.
Bonnet, G., et al. (1999) Proc. Natl. Acad. Sci. USA 96, 6171-6176.
Boutla, A., et al. (2001) Curr. Biol. 11, 1776-1780.
Braasch, D. A., and Corey, D. R. (2002) Biochemistry 41, 4503-4510.
Brantl, S. (2002a) Biochim. Biophys. Acta 1575, 15-25.
Brantl, S. (2002b) Plasmid 48, 165-73.
Brantl, S., and Wagner, E. G. (2002) J. Bacteriol. 184, 2740-2747.
Bridge, A. J., et al. (2003) Nat. Genet. 34, 263-264.
Bruice, T. W., and Lima, W. F. (1997) Biochemistry 36, 5004-19.
Burke, D. H, et al. (2002) Biochemistry 41, 6588-6594.
Cannichael, G. G. (2003) Nat. Biotechnol. 21, 371-372.
Chadwick, D. R., and Lever, A. M. (2000) Gene Ther. 7, 1362-1368.
Chartrand, P., et al. (2000) Methods Enzymol. 318, 493-506.
Christian, A. T., et al. (2001) Proc. Natl. Acad. Sci. USA 98, 14238-14243.
Crisell, P., et al. (1993) Nucleic Acids Res. 21, 5251-5255.
Crooke, S. T., ed. (2001) Antisense Drug Technology: Principles, Strategies, and Applications Marcel Dekker, Inc., New York.
Das, A. T., et al. (2004) J. Virol. 78, 2601-2605.
De Backer, M. D., et al. (2001) Nat. Biotechnol. 19, 235-241.
De Backer, M. D., et al. (2002) Curr. Opin. Microbiol. 3, 323-329.
De Backer, M. D., and Van Dijck, P. (2003) Trends Microbiol. 11, 470-478.
Deyev, S. M., et al. (1984) Immunol Let. 7, 315-319.
Dirks, R. W., et al. (2003) Methods 29, 51-57.
Doench, J. G., et al. (2003) Genes Dev. 17, 438-442.
Dynal (2000) Biomagnetic Techniques in Molecular Biology. Technical Handbook, 3^{rd} edition Dynal A S, Oslo, pp. 87-155.
Elbashir, S. M., et al. (2001) EMBO J. 20, 6877-6888.
Endo, S. et al. (2004) FEMS Microbiol. Lett. 234, 93-97.
Escude, C., et al. (1999) Proc. Nat. Acad. Sci. USA 96, 10603-10607.
Far R. K-K., and Sczakiel, G. (2003) Nucleic Acids Res. 31, 4417-4424.
Fedor, M. J. (2000) J. Mol. Biol. 297, 269-291.
Feldstein, P. A., and Bruening, G. (1993) Nucleic Acids Res. 21, 1991-1998.
George, S. T., et al. (1998) U.S. Pat. No. 5,741,679.
Goffin, C., et al. (1987) Nucleic Acids Res. 15, 8755-8771.
Goldbach, R., et al. (2003) Virus Res. 92, 207-212.
Gong, L., et al. (2000) Br. J. Haematol. 111, 351-358.
Grunweller, A., et al. (2003 ) Nucleic Acids Res. 31, 3185-3193.
Gryaznov, S., Lloyd, D. H. (1995) U.S. Pat. No. 5,817,795; U.S. Pat. No. 5,817,795 (1998); U.S. Pat. No. 6,048,974 (2000).
Guatelli, J. C. et al. (1990) Proc. Natl. Acad. Sci. USA 87,1874-1878.
Hafner, G. J., et al. (2001) Biotechniques 30, 852-856, 858, 860.
Hall, J. G. et al. (2000) Proc. Natl. Acad. Sci. USA 97, 8272-8277.
Hamada, M., et al. (2002) Antisense Nucleic Acids Drug Dev. 12, 301-309.
Harada, K., Orgel, L. E. (1993) Nucleic Acids Res. 21, 2287-229.
Hardenbol, P., et al. (2003) Nat. Biotechnol. 21, 673-638.
Heasman, J. (2002) Dev Biol. 243, 209-214.
Heim, A. et al. (1998) Nucleic Acids Res. 26, 2250-2251.
Hellyer T. J. and Nadeau, J. G. (2004) Expert Rev. Mol. Diagn. 4, 251-261.
Hertel, K. J., et al. (1998) Biochemistry 37, 16983-16988.
Ho, S. P., et al. (1996) Nucleic Acids Res. 24, 1901-1907.
Ho, S. P., et al. (1998) Nat. Biotechnol. 16, 59-63.
Hohjoh, H. (2002) FEBS Lett. 521, 195-199.
Holen, T., et al. (2002) Nucleic Acids Res. 30, 1757-1766.
Holen, T., et al. (2003) Nucleic Acids Res. 31, 2401-2407.
Jackson, A. L., et al. (2003) Nat. Biotechnol. 21, 635-637.
James, K., et al. (1998) Nucleic Acids Res. 26, 5203-5211.
Ji, Y., et al. (2001) Science 293, 2266-2269.
Ji, Y., et al. (2002) Methods Enzymol. 358: 123-8.
Jiang, M., and Milner, J. (2002) Oncogene 21, 6041-6048.
Johnston, B. H., et al. (1998) International Patent Application (PCT) W099/09045A1.
Johnston, B. H., et al. (2000) Fifth Annual Meeting of the RNA Society. May 30-June 4, Madison, Wis. Abstract Book, p. 415.
Johnston, B. H., et al. (2002) Seventh Annual Meeting of the RNA Society. May 28-June 2, Madison Wis. Abstract Book, p. 529.
Johnston, B. H., et al. (2003) Australian Patent AU756301.
Kawasaki, H., and Taira, K. (2002) Nucleic Acids Res. 30, 3609-3614.
Kazakov, S. A. (2001) In Alizadeh, B. N. (ed.), Biotechnology International (Special Millenniun ed.). Universal Medical Press, Hong Kong, Vol. III, pp. 273-281.
Kazakov, S. A., et al. (2002) International Patent Application (PCT) W003/100100 A1.
Kim, D. H., et al. (2004) Nat. Biotechnol. 22, 321-325.
Kisich, K. O., et al. (1997) Biochem. Biophys. Res. Commun. 236, 205-211.
Kisich, K. O., et al. (1999) J. Immunol. 163, 2008-2016.
Kitabwalla, M., and Ruprecht, R. M. (2002) New Engl. J. Med. 347, 1364-1367.
Komatsu, Y., et al. (1993) J. Am. Chem. Soc. 116,-3692-3696.
Komatsu, Y., et al. (1995) J. Mol. Biol. 252,296-304.
Komatsu, Y., et al. (2002) Biochemistry 41, 9090-9098.
Kramer, F. R., et al. (1997) U.S. Pat. No. 5,616,459.
Kruger, M., et al. (2000) Proc. Natl. Acad. Sci. USA 97, 8566-8571.
Kubota, K., et al. (2003) J. Virol. 77, 11016-26.
Kuhn, H., et al. (2002) Nucleic Acids Res. 30, 574-580.
Kumar, M., and Carmichael, G. G. (1998) Microbiol. Mol. Biol. Rev. 62, 1415-1434.
Kurreck, J. (2003) Eur. J Biochem. 270, 1628-1644.
Kwiatkowski, et al. (1996) Nucleic Acids Res. 24, 4632-8.
Lafarge-Frayssinet, C., et al. (1997) Cancer Gene Ther. 4, 276-285.
Landegren, U., et al. (1996) Methods 9, 84-90.
Landgraf, R., et al. (1996) Nucleic Acids Res. 24, 3246-3252.
Laptev, A. V., et al. (1994) Biochemistry 33, 11033-11039.
Lavery, K. S., King, T. H. (2003) Curr. Opin. Drug Discov. Devel. 6, 561-569.
Lesnik, E. A., Freier, S. M. (1995) Biochemistry 34, 10807-15.
Li, H., et al. (2002) Science 296, 1319-1321.
Li, W. X., and Ding, S. W. (2001) Curr. Opin. Biotechnol. 12,150-154.
Li, W. X., et al. (2004) Proc. Natl. Acad. Sci. USA. 101, 1350-1355.
Lichtenstein, C., and Nellen, W., eds. (1998) Antisense Technology. A practical Approach (The Practical Approach Series 185), Oxford University Press, Oxford-N.Y.
Lichner, Z., et al. (2003) J. Gen. Virol. 84(Pt 4), 975-980.
Lieber, A., and Strauss, M. (1995) Mol. Cell. Biol. 15, 540-551.
Lima, W. F., et al. (1997) J. Biol. Chem. 272, 626-638.
Lizardi, P. M., et al. (1998) Nat. Genet. 19, 225-232.
Lloyd, B. H., et al. (2001) Nucleic Acids Res., 29, 3664-3673.
Luo, J., et al. (1996) Nucleic Acids Res. 24, 3071-78.
Mallory, A. C., et al. (2002) Proc. Natl. Acad. Sci. USA 99, 15228-15233.
Martinez, J., et al. (2002) Cell 110, 563-574.
Matveeva, O., et al. (1997) Nucleic Acids Res. 25, 5010-5016.
McCaffrey, A. P., et al. (2003) Hepatology 38, 503-508.
Mercatante, D. R., et al. (2001) J. Biol. Chem. 276, 16411-16417.
Miller, V. M., et al. (2003) Proc. Natl. Acad. Sci. USA 100, 7195-7200.
Milner, N., et al. (1997) Nat. Biotechnol. 15, 537-541.
Miyagishi M, et al. (2003) Antisense Nucleic Acid Drug Dev. 13, 1-7.
Molenaar, C., et al. (2001) Nucleic Acids Res. 29, E89-9.
Mueller, J. D. (1997) Histochem. Cell. Biol. 108, 431-437.
Myer, S. E., Day, D. J. (2001) Biotechniques 30, 584-588, 590, 592-3.
Nagamine, K. et al. (2002) Mol. Cell. Probes 16,223-229.
Nilsson, M., et al. (1994) Science 265, 2085-2088.
Nilsson, M., et al. (2000) Nat. Biotechnol. 18, 791-793.
Nilsson, M., et al. (2001) Nucleic Acids Res. 29, 578-581.
Noonberg, S. B., et al. (1994) Nucleic Acids Res. 22, 2830-2836.
Ohmichi, T., and Kool, E. T. (2000) Nucleic Acids Res. 28, 776-783.
Olson, M. C. et al. (2004) Methods Mol. Biol. 258, 53-69.
Opalinska, J. B., and Gewirtz, A. M. (2002) Nat. Rev. Drug Discov. 1, 503-514.
Paddison, P. J., and Hannon, G. J. (2003) Curr. Opin. Mol. Ther. 5,217-224.
PailLasson, S., et al. (1997) Exp. Cell. Res. 231, 226-233.
Pan, D., et al. (1998) Bioorg. Med. Chem. Lett. 8, 1317-1320.
Pan, W. H., et al. (2001) RNA 7, 610-21.
Pasternack, R. et al. (1997) J. Clin. Microbiol. 35, 676-678.
Patzel, V., Sczakiel, G. (2000) Nucleic Acids Res. 28, 2462-2466.
Pederson, T. (2001) Nucleic Acids Res. 29, 1013-1016.
Persengiev, S. P., et al. (2004) RNA 10, 12-18.
Politz, J. C., et al. (1998) Proc. Natl. Acad. Sci. USA 95, 6043-6048.
Porta, H., and Lizardi, P. M. (1995) Biotechnology 13, 161-164.
Pritchard, C. E., and Southern, E. M. (1997) Nucleic Acids Res. 25, 3403-3407.
Probst, J. C., and Skutella, T. (1996) Biochem. Biophys. Res. Commun. 225, 861-868.
Pululdcunat, D. K., et al. (2003) Methods Mol. Biol. 236,295-310.
Pusch, O., et al. (2003) Nucleic Acids Res. 31, 6444-6449.
Qi, X., et al. (2001) Nucleic Acids Res. 29, e116.
Qu, F., et al. (2003) J. Virol. 77, 511-522.
Raap, K. et al. (1995) Human Mol. Genet. 259, 529-534.
Raj, S. M., Liu, F. (2003) Gene 313, 59-69.
Reed, J. C., et al. (2003) Virology 306, 203-209.
Roberts, R. W., Crothers, D. M. (1992) Science 258, 1463-1436.
Robertson, M. P., and Ellington, A. D. (2000) Nucleic Acids Res 28, 1751-1759.
Rossi, J. J. (1999) Chem. Biol. 6, R33-7.
Ruffner, D. E., et al. (1999) International Application (PCT) WO99/50457.
Samuel, C. E. (2004) Nat. Biotechnol 22, 280-282.
Saxena, S., et al. (2003) J. Biol. Chem. 278, 44312-44319.
Scacheri, P. C., et al. (2004) Proc. Natl. Acad. Sci. USA 101,1892-1897.
Scherr, M., et al. (2001) Mol. Ther. 4, 454-460.
Scherer, L. J., and Rossi, J. J. (2003) Nat. Biotechnol. 21, 1457-1465.
Sczakiel, G., and Far, R. K. (2002) Curr. Opin. Mol. Ther. 4, 149-153.
Sioud, M. et al. (1992). J. Mol. Biol. 223, 831-835.
Sioud, M. (1994) J. Mol. Biol. 242, 619-629.
Sioud, M. (1996) Eur J. Immunol. 26, 1026-1031.
Seyhan, A. A., et al. (2001) Nucleic Acids Res. Symp. Ser. 42, 62-63.
Seyhan, A. A., et al. (2002) J. Biol. Chem. 277,25957-62.
Shi, Y. (2003) Trends Genet. 19, 9-12.
Sierakowska, H., et al. (2000) Methods Mol Biol. 133, 223-233.
Silverman, S. K. (2003) RNA 9, 377-383.
Sledz, C. A., et al. (2003) Nat. Cell. Biol. 5, 834-839.
Spargo, C. A. et al. (1996) Mol. Cell. Probes 10, 247-256.
Sohail, M., Southern, E. M. (2000) Adv. Drug. Deliv. Rev. 44,23-34.
Soukup, G. A., and Breaker, R. R. (2000) Curr. Opin. Struct. Biol. 10, 318-325.
Southern, E., et al. (1999) Nat. Genet. 21, 5-9.
Stein, C. A. (2000) Pharmacol. Ther. 85, 231-236.
Stein, C. A., Krieg, A. M., eds. (1998) Applied Antisense Oligonucleotide Technology. Willey-Liss New York-Toronto.
Stiege, W. et al. (1988) Nucleic Acids Res. 16,4315-4329.
Stull, R. A., et al. (1996) Antisense Nucleic Acids Drug Dev. 6, 221-8.
Sullenger, B. A., and Gilboa, E. (2002) Nature. 418,252-258.
Sullivan J M, et al. (2002) Mol. Vis. 8,102-113.
Takeda, A., et al. (2002) FEBS Lett. 532(1-2), 75-79.
Tavitian, B., et al. (1998) Nat. Med. 4, 467-471.
Taylor, J. K., et al. (1999) Nat. Biotechnol. 17, 1097-1100.
Terryn, N., and Rouze, P. (2000) Trends Plant Sci. 5, 394-396.
Thomas, D. C., et al. (1999) Arch. Pathol. Lab. Med. 123, 1170-1176.
Tijsterman, M., et al. (2002) Science 295, 694-697.
Toulme, J.-J. (2001) Nat. Biotechnol. 19, 17-18.
Tuschl, T. (2002) Nat. Biotechnol. 20, 446-448.
Upegui-Gonzalez, L. C., et al. (1998) Adv. Exp. Med. Biol. 451, 35-42.
Vanhee-Brossollet C, and Vaquero C. (1998) Gene 211, 1-9.
Varga, L. V., et al. (1999) Immunol. Lett. 69, 217-224.
Vargason, J. M., et al. (2003) Cell 115, 799-811.
Vickers, T. A., et al. (2003) J. Biol. Chem. 278, 7108-7118.
Volloch, V., et al. (1991 a) Biochem. Biophys. Res. Commun. 179, 1593-1599.
Volloch, V., et al. (1991b) Biochem. Biophys. Res. Commun. 179, 1600-1605.
Wagner, E. G., et al. (2002) Adv. Genet. 46: 361-98.
Walker, G. T. et al. (1984) Nucleic Acids Res. 22, 2670-2677.
Walton, S., et al. (1999) Biotechnol. Bioeng. 65, 1-9.
Wang, E., et al. (2001) Nat. Biotechnol. 19, 371-374.
Wang, D. Y., et al. (2002) J. Mol. Biol. 318, 33-43.
Warashina M, et al. (2000) Structure Fold. Des. 8, R207-12.
Wharam, S. D. Nucleic Acids Res. 29, E54-4.
Wiber, J. C. (1997) Immunol. Invest. 26, 9-13.
Wrzesinski, J., et al. (2000) Nucleic Acids Res. 28, 1785-1793.
Wu, D. Y., and Wallace, R. B. (1989a) Genomics 4, 560-569.
Wu, D. Y., and Wallace, R. B. (1989b) Gene 76,245-254.
Wu, P., et al. (2002) Eur. J. Biochem. 269, 2821-30.
Yang, S. P., et al. (2003) Acta Pharmacol. Sin. 24, 897-902.
Yelin, R., et al. (2003) Nat. Biotechnol. 21, 379-386.
Yin, D., Ji, Y. (2002). Curr. Opin. Microbiol. 5, 330-3.
Yoshinari, K., et al. (2004) Nucleic Acids Res. 32, 691-699.
Yu, J. Y., et al. (2002) Proc. Natl. Acad. Sci. USA 99, 6047-52.
Yu, Q., et al. (1998) J. Biol. Chem. 273, 23524-23533.
Zamore, P. D. (2002) Science 296, 1265-1269.
Zeng, Y., et al. (1999) Chin. Med. J. (Engl). 112, 107-111.
Zeng, Y., et al. (2003) Proc. Natl. Acad. Sci. USA 100, 9779-9784.
Zhang, D. Y., et al. (1998) Gene 211, 277-285.
Zhang, D. Y. et al. (2001) Mol. Diagn. 6, 141-150.

We claim:

1. A polynucleotide which specifically binds to a target nucleic acid molecule and circularizes around said target, wherein said polynucleotide comprises:
a target binding sequence which is at least partially complementary and capable of binding to a sequence of the target; and
a catalytic domain which is capable of catalytic activity, wherein said catalytic activity is inhibited in the absence of binding of the polynucleotide to the target.

2. A polynucleotide according to claim 1, wherein said catalytic activity catalyzes said circularization of the polynucleotide around the target.

3. A polynucleotide according to claim 2, wherein said catalytic activity is a ligase activity.

4. A polynucleotide according to claim 3, wherein said ligase activity comprises ligation of 5′ and 3′ ends of said polynucleotide to topologically link the polynucleotide to the target.

5. A polynucleotide according to claim 3, wherein said ligase activity comprises ligation of the 5′ end of said polynucleotide to the 2′ hydroxyl group of an internal nucleotide of said polynucleotide.

6. A polynucleotide according to claim 3, wherein said catalytic domain is the catalytic domain of a hairpin ribozyme.

7. A polynucleotide according to claim 1, wherein said catalytic domain comprises ribonucleotide residues or analogs thereof.

8. A polynucleotide according to claim 1, wherein said catalytic domain comprises deoxyribonucleotide residues or analogs thereof.

9. A polynucleotide according to claim 1, wherein said catalytic domain comprises both ribonucleotide and deoxyribonucleotide residues, or analogs thereof.

10. A polynucleotide according to claim 1, wherein the inhibition of said catalytic activity is effected by a regulatory nucleic acid sequence which binds to at least a portion of the target binding sequence, thereby preventing said circularization when the target binding sequence is not bound to the target.

11. A polynucleotide according to claim 1, wherein said target comprises RNA.

12. A polynucleotide according to claim 1, wherein said target comprises DNA.

13. A polynucleotide according to claim 1, wherein said polynucleotide is prepared synthetically.

14. A polynucleotide according to claim 1, wherein said polynucleotide is prepared by expression from an expression vector.

15. A polynucleotide according to claim 14, wherein said expression occurs in vitro.

16. A polynucleotide according to claim 14, wherein said expression occurs in vivo.

17. A polynucleotide according to claim 16, wherein said polynucleotide is expressed by RNA polymerase II or III in the nucleus of a host cell.

18. A complex comprising a polynucleotide according to claim 1 circularized around said target molecule.

19. A method for circularizing a polynucleotide around a target nucleic acid molecule, said method comprising contacting said target molecule with a polynucleotide according to claim 1, wherein binding of said target binding sequence to said target prevents inhibition of said catalytic activity, thereby allowing circularization to proceed.

20. A method for reducing efficiency of transcription, comprising topologically linking a polynucleotide to a target according to the method of claim 19, wherein said topological linkage reduces efficiency of transcription from the target.

21. A method for reducing efficiency of translation, comprising topologically linking a polynucleotide to a target according to the method of claim 19, wherein said topological linkage reduces efficiency of translation from the target.

22. A method for detecting presence or absence of a target nucleic acid molecule, said method comprising contacting a composition suspected of containing said target with a polynucleotide according to claim 1 and detecting circularization of the polynucleotide around the target, wherein presence of said circularization indicates presence of the target in the composition, if any.

23. A method according to claim 22, wherein said target is linked to a solid support.

24. A method according to claim 23, wherein said solid support is a hybridization membrane.

25. A method according to claim 22, wherein said polynucleotide is comprised within an array.

26. A method according to claim 22, wherein said detection comprises amplification of the circularized polynucleotide.

27. A method according to claim 26, where said amplification comprises rolling circle amplification.

28. A method according to claim 22, wherein said polynucleotide comprises a detectable label and said detection comprises detection of the label bound to the target.

29. A method according to claim 28, wherein said label is selected from the group consisting of radioactive, fluorescent, hapten, or enzymatic labels, or a member of a binding pair.

30. A library comprising a plurality of polynucleotides, wherein each of said polynucleotides comprises a target binding sequence, a catalytic domain which is capable of catalytic activity, and a regulatory sequence which inhibits catalytic activity in the absence of binding between the target binding sequence and a nucleic acid target, and wherein at least one of the target binding sequence, the catalytic domain, and the regulatory sequence is at least partially randomized.

31. A method for selection of polynucleotides that are capable of topologically linking to a target nucleic acid molecule, comprising contacting said target with a plurality of polynucleotides from a library according to claim 30, and amplifying the polynucleotides which become topologically linked to the target.

32. A kit comprising a polynucleotide according to claim 1.

33. A kit comprising a library according to claim 30.
